SEQUENCE LISTING (78 sequences)

SEQ ID NO: 1 - DNA, 20 bp, Homo sapiens
cgagatccctccaaaatcaa

SEQ ID NO: 2 - DNA, 20 bp, Homo sapiens
ggcagagatgatgacccttt

SEQ ID NO: 3 - DNA, 21 bp, Homo sapiens
tttgtgggcctgaagaaaact

SEQ ID NO: 4 - DNA, 21 bp, Homo sapiens
agggctgtcctgaataagcag

SEQ ID NO: 5 - DNA, 20 bp, Homo sapiens
gtggaggaagctgacaacaa

SEQ ID NO: 6 - DNA, 20 bp, Homo sapiens
attctccaggttgcctctca

SEQ ID NO: 7 - DNA, 20 bp, Homo sapiens
ccagcagacttcacatgtcc

SEQ ID NO: 8 - DNA, 20 bp, Homo sapiens
acatgtgtgagaggggcagt

SEQ ID NO: 9 - DNA, 20 bp, Homo sapiens
gagccttcaggtcacagagc

SEQ ID NO: 10 - DNA, 20 bp, Homo sapiens
tccacacagggggtgtactt

SEQ ID NO: 11 - DNA, 20 bp, Homo sapiens
aagtggatgctccagtccat

SEQ ID NO: 12 - DNA, 20 bp, Homo sapiens
atccaagggcctagttcgag

SEQ ID NO: 13 - DNA, 20 bp, Homo sapiens
ggagttccagccatgctctt

SEQ ID NO: 14 - DNA, 20 bp, Homo sapiens
cctgcttgaagatgggcaga

SEQ ID NO: 15 - DNA, 20 bp, Homo sapiens
cccaattacccatccttcct

SEQ ID NO: 16 - DNA, 19 bp, Homo sapiens
caggtgtgccttgagatgg

SEQ ID NO: 17 - DNA, 23 bp, Homo sapiens
caaagtgagacctgccaaaaaga

SEQ ID NO: 18 - DNA, 23 bp, Homo sapiens
tgggacaagggaatctgacagtg

SEQ ID NO: 19 - DNA, 19 bp, Homo sapiens
catgcacggtccacacctc

SEQ ID NO: 20 - DNA, 21 bp, Homo sapiens
ctcatggaccttcatgtgctt

SEQ ID NO: 21 - DNA, 20 bp, Homo sapiens
cgattccgctacgaccaact

SEQ ID NO: 22 - DNA, 20 bp, Homo sapiens
gtttgagggggaatgctgga

SEQ ID NO: 23 - DNA, 20 bp, Homo sapiens
ttcctccgaccccctaacaa

SEQ ID NO: 24 - DNA, 20 bp, Homo sapiens
gataagtggcgttggcttgc

SEQ ID NO: 25 - DNA, 22 bp, Homo sapiens
tctcataaccctcaacacccac

SEQ ID NO: 26 - DNA, 21 bp, Homo sapiens
aggcggcaaagactagtatgg

SEQ ID NO: 27 - DNA, 21 bp, Homo sapiens
ctgtaagtggttcaacgtgcg

SEQ ID NO: 28 - DNA, 21 bp, Homo sapiens
ccatgtgcagcttactctggt

SEQ ID NO: 29 - DNA, 19 bp, Homo sapiens
cccaccacgtacaagggtc

SEQ ID NO: 30 - DNA, 19 bp, Homo sapiens
ctggggtattgggggcatc

SEQ ID NO: 31 - DNA, 21 bp, Homo sapiens
aacagggttccagcacagcag

SEQ ID NO: 32 - DNA, 20 bp, Homo sapiens
ggccagacacattccagcaa

SEQ ID NO: 33 - DNA, 20 bp, Homo sapiens
gggagccactgagaacagag

SEQ ID NO: 34 - DNA, 20 bp, Homo sapiens
tggctcctaaatccatcctg

SEQ ID NO: 35 - DNA, 18 bp, Homo sapiens
acgccgagttgagcaaga

SEQ ID NO: 36 - DNA, 18 bp, Homo sapiens
tctgcctcctccacgaag

SEQ ID NO: 37 - DNA, 20 bp, Homo sapiens
gcaaaaatacttcccccaca

SEQ ID NO: 38 - DNA, 18 bp, Homo sapiens
tctcccgcaccagtcatc

SEQ ID NO: 39 - DNA, 20 bp, Homo sapiens
gtgccaagctcagggtgtag

SEQ ID NO: 40 - DNA, 20 bp, Homo sapiens
tccaacaggcctgagaaatc

SEQ ID NO: 41 - DNA, 19 bp, Homo sapiens
ctgttggagacctggatgc

SEQ ID NO: 42 - DNA, 20 bp, Homo sapiens
cgtcagttgtcccttgtcac

SEQ ID NO: 43 - DNA, 20 bp, Homo sapiens
ggaaaagatctggcaccact

SEQ ID NO: 44 - DNA, 20 bp, Homo sapiens
gagtcattttctcccggttg

SEQ ID NO: 45 - DNA, 19 bp, Homo sapiens
ccaccattttcccaggtct

SEQ ID NO: 46 - DNA, 19 bp, Homo sapiens
attttccccgaggcactct

SEQ ID NO: 47 - DNA, 21 bp, Homo sapiens
tttcatctttggtcagagtgg

SEQ ID NO: 48 - DNA, 21 bp, Homo sapiens
tcgcagttttcacactccttc

SEQ ID NO: 49 - DNA, 19 bp, Homo sapiens
cgtggtcaaaccaatcgac

SEQ ID NO: 50 - DNA, 20 bp, Homo sapiens
aacagacacggttgcagttg

SEQ ID NO: 51 - DNA, 21 bp, Homo sapiens
gctgacaccagacttgggttt

SEQ ID NO: 52 - DNA, 20 bp, Homo sapiens
cccctcgagcaaagaaaacg

SEQ ID NO: 53 - DNA, 21 bp, Homo sapiens
gacagagcggaaaagtgggaa

SEQ ID NO: 54 - DNA, 21 bp, Homo sapiens
ccttctccctcagctgatctt

SEQ ID NO: 55 - DNA, 21 bp, Homo sapiens
acctcaacagctccctgactc

SEQ ID NO: 56 - DNA, 23 bp, Homo sapiens
ataatcgccgccacaaactctcc

SEQ ID NO: 57 - DNA, 20 bp, Homo sapiens
ggcggcacgaaatatcctct

SEQ ID NO: 58 - DNA, 20 bp, Homo sapiens
ggaggcgagcatctcctttt

SEQ ID NO: 59 - DNA, 21 bp, Homo sapiens
cccctgctatttcatcgaccc

SEQ ID NO: 60 - DNA, 20 bp, Homo sapiens
gacacacggctccacttgat

SEQ ID NO: 61 - DNA, 21 bp, Homo sapiens
aggagaaacagggcctacaga

SEQ ID NO: 62 - DNA, 19 bp, Homo sapiens
ggagggtcctgtacgtggc

SEQ ID NO: 63 - DNA, 20 bp, Homo sapiens
aggaggaggttcgggaactc

SEQ ID NO: 64 - DNA, 20 bp, Homo sapiens
tcatactgcgtgcggatctc

SEQ ID NO: 65 - DNA, 20 bp, Homo sapiens
cacgagacccactttttccg

SEQ ID NO: 66 - DNA, 21 bp, Homo sapiens
accagaccttgacctgactct

SEQ ID NO: 67 - DNA, 20 bp, Homo sapiens
ctccacgaagaggaaatcca

SEQ ID NO: 68 - DNA, 20 bp, Homo sapiens
gtgaggtcaggcttggaaac

SEQ ID NO: 69 - DNA, 22 bp, Homo sapiens
tggacatagctgatgtggctta

SEQ ID NO: 70 - DNA, 20 bp, Homo sapiens
ccaggatccagatccagttg

SEQ ID NO: 71 - DNA, 20 bp, Homo sapiens
caaggcctcagtcagtgtga

SEQ ID NO: 72 - DNA, 20 bp, Homo sapiens
ccggaaagctgtagatggtc

SEQ ID NO: 73 - DNA, 22 bp, Homo sapiens
accccacacgcctttggcacaa

SEQ ID NO: 74 - DNA, 24 bp, Homo sapiens
cacacccctggaataagccgagct

SEQ ID NO: 75 - DNA, 23 bp, Homo sapiens
tttgatagatgctgacccatacc

SEQ ID NO: 76 - DNA, 20 bp, Homo sapiens
atgctgtggtgctttgaggt

SEQ ID NO: 77 - DNA, 20 bp, Homo sapiens
ccagagctgtgcagatgagt

SEQ ID NO: 78 - DNA, 20 bp, Homo sapiens
gcatttgtggttgggtcagg

(19) United States
(12) Patent Application Publication
(10) Pub. No.: US 2024/0247235 A1
(43) Pub. Date: Jul. 25, 2024

(54) niPSCs DERIVED FROM SOMATIC CELLS

(71) Applicants: UNIVERSITA' DEGLI STUDI DI PADOVA, Padova (IT); FONDAZIONE TELETHON, Roma (IT)

(72) Inventors: Graziano MARTELLO, Padova (IT); Nicola ELVASSORE, Padova (IT); Marco PELLEGRINI, Bassano del Grappa, Vicenza (IT); Stefano GIULITTI, Istrana, Treviso (IT)

(21) Appl. No.: 16/972,379

(22) PCT Filed: Jun. 6, 2019
(86) PCT No.: PCT/IB2019/054718

§ 371 Date: Dec. 4, 2020

(30) Foreign Application Priority Data
Jun. 6, 2018 (IT) 102018000006076

(51) Int. Cl. C12N 5/074

(52) U.S. Cl. CPC C12N 5/0696; C12N 2501/602; C12N 2501/603; C12N 2501/604; C12N 2501/605; C12N 2501/606; C12N 2501/727; C12N 2506/1307

(57) ABSTRACT

A method obtains naive induced iPSCs (niPSCs) from mammalian somatic cells. The method includes a transgene cocktail delivery to somatic cells in a microfluidic setting, including a first step of Mesenchymal to Epithelial Transition (MET) and a second step of colonies generation.

The present invention finds application in the medical field and, in particular, to the preparation of naïve induced Pluripotent Stem Cells (niPSCs).

### BACKGROUND

Conventional human PSCs (Pluripotent Stem Cells), either derived from early embryos (Thomson, J A et al. Embryonic Stem Cell Lines Derived from Human Blastocysts. Science 1998, 282, 1145-1147) or by reprogramming of somatic cells by expression of transcription factors OCT4, SOX2, KLF4 and cMYC (OSKM) (Takahashi, K. et al. Induction of pluripotent stem cells from adult human fibroblasts by defined factors. Cell 2007, 131, 861-872), resemble a distinct, more advanced developmental stage, named primed pluripotency.

Primed PSCs express only OCT4, SOX2 and NANOG in response to FGF and TGFbeta signals, display higher levels of repressive epigenetic modifications and are mostly glycolytic (Nichols, J & Smith, A Naive and Primed Pluripotent States. Cell Stem Cell 2009, 4, 487-492; Hackett, J A & Surani, MA Regulatory Principles of Pluripotency: From the Ground State Up. Cell Stem Cell 2014, 15, 416-430). Human primed PSCs are also heterogeneous and different lines display a differentiation bias toward certain germ layers (Osafune, K et al. Marked differences in differentiation propensity among human embryonic stem cell lines. Nat. Biotechnol. 2008, 26, 313-315).

niPSCs cells have been defined for instance in the international patent application WO 2016/027099 (Cambridge Entpr Ltd, GB).

Several protocols have been developed so far, whereby human naïve PSCs are generated either by expression of transgenes together with genomic reporter constructs or directly from human embryos (Weinberger, L et al. Dynamic stem cell states: naive to primed pluripotency in rodents and humans. Nat. Rev. Mol. Cell Biol. 2016, 17, 155-169; Davidson, K C et al. The pluripotent state in mouse and human. Development 2015, 142, 3090-3099; Ware, C B Concise Review: Lessons from Naïve Human Pluripotent Cells. STEM CELLS 2017, 35, 35-41). These studies showed that human naïve PSCs can be generated, yet they are not as commonly utilized as conventional primed PSCs.

The use of human embryos has ethical limitations and it obviously does not allow generation of patient-specific naïve PSCs; on the other hand, conversion of somatic cells to naïve pluripotency with available protocols requires one or more rounds of stable genetic manipulations that are time-consuming, inefficient, and potentially mutagenic.

Liu et al. in “Comprehensive characterization of distinct states of human naive pluripotency generated by reprogramming” (Nat Methods 2017, 14 (11): 1055-1062) describe obtaining human naïve iPSCs by viral infection of human fibroblasts. The drawback associated with said methodology is that the virus, although a Sendai, non-integrating virus, remains in the niPSCs for months after the infection; moreover, the procedure is time consuming, wherein colonies are obtained only after 20-24 days. In addition, data are not available with respect to efficiency.

Luni et al. in “High-efficiency cellular reprogramming with microfluidics” (Nat Methods 2016, 13 (5): 446-52) describe obtaining iPSCs from different sources of human somatic cells; however, the obtained cells are primed iPSCs, expressing OCT4, NANOG and SSEA-4.

Therefore, there is a strong need to find methods to generate human naïve PSCs (niPSCs) in an accessible and efficient manner.

### SUMMARY OF THE INVENTION

The authors of the present invention have surprisingly found that naïve iPSCs (niPSCs) may be obtained from mammalian somatic cells, with a method comprising a transgene cocktail delivery to somatic cells in a microfluidic setting, characterized by a first step of Mesenchymal to Epithelial Transition (MET) and a second step of colonies generation.

### OBJECT OF THE INVENTION

In a first object, the invention discloses a method to obtain naïve i PSCs (niPSCs) from somatic cells comprising a first step of Mesenchymal to Epithelial Transition (MET) and a second step of colonies generation, with a transgene cocktail delivery to somatic cells in a microfluidic cells.

The cells obtained according to the method disclosed represent a second object of the invention.

### BRIEF DESCRIPTION OF THE FIGURES

FIG. 1: schematic diagram of an embodiment of the method according to the present invention.

FIG. 2: an embodiment of a microfluidic chip design for reprogramming according to the present description. Each chip is built on common microscope glass slides and can be placed in a standard Petri dish with a phosphate buffer bath to ensure a sterile humidified environment. Fresh medium is dispensed using a standard micropipette through the inlet of the microfluidic channel. The exhausted medium is then collected from the outlet on the other end of the channel.

FIG. 3: (a) BJ fibroblasts were reprogrammed with the method according to the present invention in microfluidic (μF) channel of different heights. At day 12, immunostaining for KLF17 and POU5F1 was conducted and colonies were scored. In black double positive colonies are shown, in grey the colonies identified by their naïve-like morphology. The percentages of double positive colonies out of total colonies at different heights is indicated. (b) The number of colonies for each microfluidics channel is indicated as a dot. Mean of each condition is indicated as a black bar. n=2 biological experiments. (c) Success rate, measured as the percentage of microfluidic channels containing naïve-like colonies. Each bar indicates an independent experiment, the number above each bar indicates the number of microfluidic channels (i.e. technical replicates) used in each experiment. (d) Efficiency of generation of naïve-like colonies calculated as the number of colonies observed at day 12 per 100 cells seeded. The number of colonies for each microfluidics channel is indicated as a dot. Mean of each condition is indicated as a black bar. n=2 biological experiments. (e) Addition of LIN28 mmRNA to the OSKMN cocktail in 2iLGo+KSR+Ri in hypoxia causes a marked reduction in efficiency.

FIG. 4: (a) From day 5, 3 different naïve media have been used: 2iLGo−KSR+Ri, 4iLA and RSeT. At day 12 immunostaining for the naïve marker KLF17 and for POU5F1/OCT4 was performed and colonies were counted. Each dot represents a technical replicate, black bars indicate means. In black the double positive colonies are shown, in grey all the colonies identified by their morphology; (b) Quantification of colony size by measure of the equivalent diameter. For each condition, at least 30 colonies (dots) from 5 technical replicates were quantified. Means=black bars; (c) efficiency of niPSC generation from different somatic cells; (d) Clonal assay performed by plating 2000 cells in a well of a 12 well plate with feeder cells. HPD01 niPSCs in RSeT display high clonogenic capacity, comparable to established Reset H9 cells in 2iLGo 21, which served as positive control; (e) DNA content assessed by propidium iodide and cytofluorimetric analysis. Only HPD01 (p10) and HPD07 (p23) showed a preponderant tetraploid population after several passages in conventional culture conditions (CCC) and RSeT. Six passages after sorting (p16), HPD01 showed a diploid-like profile ad was used thereafter for other characterizations; (f) Left: Q-banding showing normal karyotype in HPD01 line at passage 19. Right: table summarising karyotyping of 5 niPSC lines; (g) Gene-expression analysis by qPCR of niPSCs. Expression relative to Primed H9 ES cells was calculated. GAPDH served as loading control.

FIG. 5: (a) Heatmap of unsupervised hierarchical clustering based on somatic and pluripotency associated markers. The naïve (black) and primed (white) iPSCs generated in this study cluster together with previously characterised human ESCs. Values displayed correspond to the gene expression level (normalized log 2 pseudo-counts) scaled by the row mean; (b) Heatmap of unsupervised clustering based on Transposable Elements expression in primed and naïve PSCs. Values displayed are transposon expression level (normalized log 2 pseudo-counts) scaled by row mean; (c) Principal components analysis (PCA) of RNA-sequencing samples. Inset shows naïve PSC lines derived in this study. As an internal control, the transcriptome from Reset H9 cells cultured in parallel to niPSCs was sequenced; (d-f) Gene-expression analysis by qPCR of reprogramming trajectories in microfluidics. (d) Fibroblasts were plated at the same density and transfected with OSKMN either in PRM (grey) to generate primed iPSCs, or in conditions allowing niPSCs formation (light grey). Samples were collected at Day 1, 5, 8, 10 and 12 and the expression of pluripotency markers was analysed, PCA analysis shows that reprogramming towards primed and naïve pluripotency clearly diverge at day 8; (e) Expression of the indicated markers during reprogramming to either naïve or primed pluripotency (grey and black, respectively). Expression relative to the highest value was calculated, n=2. GAPDH served as loading control; (f) Reduced expression of the Mesenchymal markers (black) and gradual increase in Epithelial (grey) markers at day 5 and 8 or reprogramming. Expression relative to the highest value was calculated. Mean and s.d., n=2. GAPDH served as loading control.

FIG. 6: (a) Expression of known regulators of DNA methylation in primed and naïve PSCs measured by RNA-seq; (b) Average genomewide CG methylation levels measured by RRBS in somatic (grey), primed (black) and naïve (white) PSCs; (c) Methylation profiles from RRBS analysis showing the distribution of modifications from non-methylated (0) to hyper-methylated regions (1). Both fibroblasts and primed HPD00 show a bimodal distribution, with a large fraction of hyper-methylated regions (>0.8). In contrast, niPSCs show only a small portion of hypermethylated regions; (d) Unsupervised hierarchical clustering of the genomewide methylation pattern separates niPSCs from somatic and primed cells; (e) Violin plots showing DNA methylation levels on 52 promoters identified as hypomethylated in preimplantation blastocysts relative to primed hESCs. Box plots shows medians and first and third quartiles; (f) Methylation levels at imprinted loci. Maternal, or Paternal, loci are those where the maternal, or the paternal, alleles are methylated. Placental loci are characterized by transient methylation of the maternal allele in oocytes and blastocysts that is then lost in somatic cells. Each dot indicates the average methylation level at an imprinted locus. Black bars indicated the mean methylation levels of each class of loci. Somatic and primed cells show robust methylation at Maternal and Paternal loci, while niPSCs retain only partial methylation at Paternal loci; (g) SNPs analysis of the MEG3 transcript reveals monoallelic expression in BJ fibroblasts and biallelic expression in 3 isogenic niPSC lines; (h) Violin plots show methylation levels at CpG island on X chromosome and at chromosome 19. Methylation on Chr. 19 is reduced to the same extent in male and female niPSCs, compared to fibroblasts. Methylation on Chr. X is very low in both male cells and female niPSCs, compared to female fibroblasts; (i) SNPs analysis of X-linked genes. Top: histograms showing the fraction of SNPs expression. Female fibroblasts show monoallelic, while female niPSCs show biallelic expression. Bottom: vertical bars indicate the position of the SNPs analysed on the X chromosome. (j) Expression levels measured by RNAseq of XIST. Female niPSCs express high levels of XIST. The Y-linked gene USP9Y is shown as a control.

FIG. 7: (a) Gene-expression analysis by qPCR of the indicated PSCs lines for 3 transcripts of the mitochondrial genome. Expression relative to Primed H9 ES cells was calculated. GAPDH served as loading control. Mean and s.d., n=2 (b) Analysis by RNAseq of the indicated mitochondrial transcripts in fibroblasts and niPSCs. Expression was normalised to BJ fibroblasts.

FIG. 8: (a) Strategy to compare the differentiation efficiency of niPSCs and primed iPSCs. Starting from BJ human fibroblasts isogenic primed (HPD00) or naïve PSCs (HPD01/3/4) were generated using mmRNAs in μF. Next, the EB differentiation protocol extended to 50 days was applied and the expression of markers of the three germ layers was measured by qPCR. Early, mid and late markers are shown in different shades of colours. For each preparation, at least 15 EBs that were >0.5 mm in size were pooled. Data are expressed as log 2 fold change relative to the highest value. GAPDH served as loading control; (b) qPCR analysis of EBs after 50 days of differentiation. Expression relative to the highest value was calculated. Mean and s.d., n=2. GAPDH served as loading control.

FIG. 9: (a) Comparison of efficiency of the method according to the present invention in microfluidics vs CCC. The number of naïve colonies generated for 100 cells seeded is shown. Each dot represents an independent technical replicate. Mean=black bars. (b) niPSCs generated in CCC (HPD05) show an expression profile comparable to microfluidic-derived niPSCs and previously derived naïve PSCs. Quantification by qPCR relative to Primed H9 hESCs. GAPDH served as a loading control. (c) Transcriptome analyses by RNAseq of isogenic niPSCs generated in CCC or μF, and primed iPSCs. Well-generated niPSCs are highly similar (Pearson Correlation coefficient R=0.980) to niPSCs generated in microfluidics, and distinct from primed iPSCs. Naïve- (grey) and primed-specific genes (black) are depicted.

### DETAILED DESCRIPTION OF THE INVENTION

### Definitions

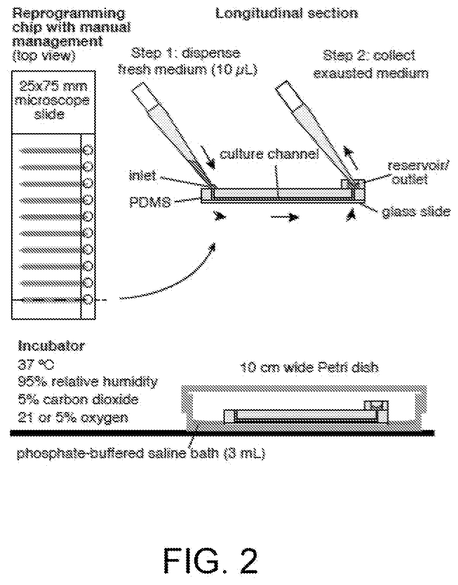

In the present description, the term “microfluidic setting” refers to the microfluidic platform described in Luni et al. 2016 (cited), Giulitti et al. 2013 (Optimal periodic perfusion strategy for robust long-term microfluidic cell culture. Lab. Chip 13, 4430), where it was found that downscaling mmRNA reprogramming to microliter volumes generates a favorable environment for the acquisition of pluripotency. Briefly, said microfluidic platforms are fabricated according to standard soft-lithographic techniques and molded in polydimethylsiloxane (PDMS). Preferably, the channels are about 45×220 μm rectangular section and culture channel mold has about 2000×220 μm rectangular section (width×high). The channels may also be 1-2 mm width and normally are 1.5 mm width (for reducing the volume of the culture medium). The microfluidic platform is preferably fully assisted by an automated medium delivery and distribution system into the culture channels. In FIG. 2 there is depicted a representative scheme of a microfluidic setting.

“Conventional culture conditions (CCC)” refers to culture conditions in standard plates, for example 100 mm petri dishes, 12, 24, or 48 well microplates.

The term “transgene cocktail” in the present description refers to a preparation comprising at least one gene to which cells are exposed.

In a preferred embodiment of the invention, said “transgene cocktail” comprises at least one nucleic acids sequence encoding a gene selected from the group consisting of: OCT4, SOX2, KLF4 and cMYC (the four being referred also as OSKM)+NANOG; or combination thereof.

Said at least one gene is vehiculated to said cells according to one of the method known in the art.

In a preferred embodiment, said “transgene cocktail” is vehiculated to said cells via mmRNAs, synthetic mRNAs prepared in vitro and modified with 3′ and 5′ UTR (Untranslated Regions) elements that increase their stability (Schlaeger, T. M. et al. A comparison of non-integrating reprogramming methods. Nat. Biotechnol. 33, 58-63 (2014)).

In a further embodiment, said “transgene cocktail” is vehiculated to said cells via viral infection.

As used herein, a “primed reprogramming medium” (PRM), suitable for generation of primed PSCs, is intended to be any eukaryotic cell culture medium comprising at least one growth factor and at least one GSK inhibitor.

In a preferred embodiment, said growth factor is FGF-2.

Preferably, the PRM of the invention comprises in addition a Rock inhibitor.

A PRM of particular interest for the present invention is StemMACS ReproBREW XF (Miltenyi biotech).

As an alternative, Essential 8 medium (E8, Chen et al., Nat Methods. 2011 May; 8 (5): 424-429), which consists of DMEM/F12 (Dulbecco's Modified Eagle Medium Nutrient Mixture F-12, Gibco), L-ascorbic acid, sodium selenium, NaHCO₃, Transferrin, Insulin, FGF₂, L-glutamine, and/or TGF-β1.

As used herein, a “medium for naïve pluripotent cells” is intended to be a cell culture medium optimized for PSC culture, suitably a naïve supporting medium, preferably a N2B27 based medium (described in Cold Spring Harb Protoc; 2017; doi:10.1101/pdb.rec096131) containing a MEK inhibitor, as an example PD0325901, a GSK inhibitor, as an example CHIR99021, a PKC inhibitor, as an example Gö6983, LIF, as an example human LIF, alternatively, an FGF-free medium, as an example the RSeT medium by Stemcell technologies Inc.

According to a first object of the invention, a method is described herein comprising the delivery of a transgene cocktail to mammalian somatic cells in a microfluidic setting.

In particular, the method comprises a first step of Mesenchymal to Epithelial Transition (MET) and a second step of colonies generation.

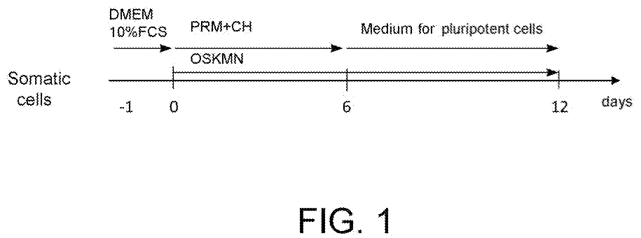

A diagrammatic representation of an embodiment of said method is represented in FIG. 1.

For the purposes of the present invention, the first step comprises:
incubating said mammalian somatic cells in a Primed Reprogramming Medium (PRM) for 5 to 8 days, from day 0 to day 5-8.

In particular, said first step is carried out in the presence of a GSK inhibitor, which is preferably represented by a GSK3A/GSK3B inhibitor.

For the purposes of the present invention, the second step comprises:
applying to the culture a medium for naïve pluripotent cells, said second step being protracted for a period of between 4 and 10 days, from day 5-8 to day 9-18, i.e. after the first step is concluded.

In a preferred embodiment, the mammalian somatic cells are human somatic cells, which are preferably represented by human fibroblast cells and still more preferably selected from the group comprising: human foreskin fibroblasts HFF and lung fibroblasts WI-38 and IMR-90.

Alternatively, said somatic cells are represented by epithelial cells and preferably by tubular kidney cells.

In another alternative embodiment, said somatic cells are represented by blood cells, preferably monocytes.

According to the present invention, every day during each of said first and said second step, a transgene cocktail is delivered to the cells, said transgene cocktail comprising at least one of the factors selected in the group consisting in: OCT4, SOX2, KLF4, CMYC, NANOG.

In a preferred embodiment, at least two of said factors are delivered to the cells.

In a still more preferred embodiment, at least two of said factors are delivered to the cells, together with an additional factors.

In a still preferred embodiment, OCT4, SOX2, KLF4 and cMYC (OSKM) are delivered, more preferably OSKM+NANOG.

In a preferred embodiment, the invention transgene cocktail is delivered via mmRNAs transfection.

Cells are preferably transfected with mmRNA for OSKMN (OSKM+NANOG).

In a preferred embodiment, the invention transgene cocktail is administered with stoichiometry 3:1:1:1:1; therefore, the amount of the OCT4 factor is preferably three times the amount of each one of the other factors.

According to the method of the invention, the somatic cells for reprogramming are seeded, preferably in DMEM+10% FBS, preferably on day −1, wherein −1 indicates one day before the start of the first step, at 15-30 cells/mm², preferably at 25 cells/mm².

When the somatic cells are plated at a lower density, it has been observed that niPSCs do not generate.

At day 0, the first step of the invention starts and the medium is changed to PRM medium, according to the above definition.

Preferably, every day said PRM medium is changed with fresh medium and mmRNAs are added about 12-17 h, preferably about 15 h, after medium change.

The transfection is continued for about 4 to 12 h, preferably for about 9 h.

Said GSK3 inhibitor is preferably selected in the group comprising: CHIR99021 (Stemcell technologies Inc.), SB-216763 (BIOMOL International), IM-12 (Enzo).

In a preferred embodiment, 1 μM CHIR99021 is added.

In a further preferred embodiment of the invention, during the first step of the method a ROCK inhibitor is also added.

Preferably, said ROCK inhibitor is Y-27632 (Sigma Aldrich).

Preferably, an amount of 5 μM Y-27632 is added.

According to the present invention, in the second step, said medium for naïve pluripotent cells is selected from the group comprising: RSeT (Stemcell technologies Inc), 4iLA (Theunissen T W et al. “Systematic identification of culture conditions for induction and maintenance of naïve human pluripotency” Cell Stem Cell. 2014, 15:471-487) and 2iLGo (Takashima, Cell. 2014 Sep. 11; 158 (6): 1254-1269).

During the second step of the invention method, the medium is changed with fresh medium every day, and said mmRNA is added every day, according to the same time scheme above reported for said first step.

Therefore, the mmRNAs are added about 12-17 h, preferably about 15 h, after medium change and the transfection is continued for about 4 to 12 h preferably for about 9 h.

In a preferred embodiment, the cells are maintained, at least for a fraction of the duration time of the method, in a 5% CO₂, 5% O₂ incubator, still more preferably cells are maintained in a 5% CO₂, 5% O₂ incubator during said first and said second step.

In another preferred embodiment, during said first and second steps the cells grow on an extracellular matrix, preferably represented by fibronectin, matrigel or laminin.

In a still further embodiment of the invention, during said first and second steps the B18R protein, a type I interferon (IFN)-binding protein, is added to suppress single-strand-RNA-induced immune response mediated by type I interferons.

According to a preferred embodiment of the invention, the first step is protracted for 6 days and the second step for 6 days.

For the purposes of the present invention, the method described is performed within a microfluidic setting.

Advantageously, an amount of only ˜200 μl of medium is used during a 14-day reprogramming method for each channel.

Typically, each chip comprises 5-15 channels.

The mRNA technology allows the rapid attainment of transgene-free niPSCs and, due to the short lifespan of transfected mmRNA, the cells can be readily differentiated at the end of the reprogramming protocol within the same system.

Advantageously, the method according to the present invention does not require a feeder-layer.

The colonies obtained according to the method described herein have demonstrated to be expandable, whilst displaying a stable naïve phenotype; in addition, the cells obtained show to be highly clonogenic and have a high chromosomal stability.

Is has been surprisingly found that the cells obtained according to the method of the present invention express the naïve markers KLF17, TFCP2L1, DPPA3 and DNMT31L, while they do not express SSEA4.

Also, the obtained cells show low DNA methylation levels and an high mitochondrial activity.

### Experimental Data

### Methods

### Cell Culture

Naïve human embryonic stem cells (Reset H9, Takashima et al. 2014 Resetting Transcription Factor Control Circuitry toward Ground-State Pluripotency in Human. Cell 158, 1254-1269) were cultured on mitotically-inactivated mouse embryonic fibroblasts (MEF, DR4 ATCC) in 2iLGo medium prepared as follow: 18 N2 and 2% B27 supplements in DMEM-F12: Neurobasal (N2B27 medium, all Thermo scientific) were supplemented with 1 μM PD0325901 (PD, Axon Medchem), 1 μM CHIR99021 (CH, Axon Medchem), 10 ng ml⁻¹ human LIF, 1-2 μM Gö6983 (Go, Axon Medchem).

It was particularly critical to titrate the concentration of each batch of Gö6983 in order to minimize cell stress due to accumulation of the inhibitor and maximize expression of naïve markers. Naïve human iPSCs (niPSCs) were cultured in various naïve supporting media reported in Table 1 on MEF feeders.

TABLE 1
naïve supporting media

| | |
|---|---|
| N2B27 basal | 1:1 Neurobasal medium:DMEM-F12, 2% N2, 2% B27, |
| | 1% L-glutamine, 0.1 mM 2-mercaptoethanol (all Thermo) |
| 2iLGo | N2B27 basal, 1 μM PD03, 1 μM CH, 1-2 μM Go |
| | (batch titration required), 10 ng ml⁻¹ LIF |
| 4iLA | N2B27 basal, 1 μM PD03, 1 μM WH-4-023 |
| | (Axon Medchem), 0.5 μM SB590885 (Axon Medchem), |
| | 10 μM Ri, 10 ng ml⁻¹ LIF, 20 ng ml⁻¹ Activin-A |
| RSeT | Basal medium supplemented with 5×, 500×, and |
| | 1000× supplements (05970, StemCell technologies). |
| | FGF-free medium. |

niPSC colonies were passaged every 4-6 days as follows. Cells were washed with phosphate buffer without Ca²⁺/Mg²⁺ (PBS) and incubated with 300 μl TripLE select (Thermo Scientific) per 12-well plate for 3 min at room temperature. N2B27 medium (700 μl) was added to inhibit dissociation. Clusters (3-5 cells) were obtained by pipetting twice the entire volume. Cell suspensions were centrifuged at 300 g for 4 min, resuspended in naïve medium with 10 μM Y27632 Rho-associated kinase (ROCK) inhibitor (Ri, Axon Medchem), and seeded on ˜300 MEF per mm². ROCK inhibitor was used only for 24 h after passaging.

BJ (passage 12) and HFF-1 (passage 18) human foreskin fibroblasts (ATCC) were cultured in DMEM with 10% fetal bovine serum (FBS, Sigma-Aldrich) before reprogramming. Somatic cells were cultured in normoxia (21% O₂, 5% CO₂, 37° C.), pluripotent stem cells were cultured in hypoxia (5% O₂, 5% CO₂, 37° C.) with daily medium changes. All cell lines were mycoplasma-negative (Mycoalert, Lonza).

### Microfluidic Chips Production

Microfluidics chips were produced as previously reported (Giulitti et al. 2013, cited; Luni et al. 2016, cited). Briefly, Sylgard 184 (Dow Corning) was cured on a 200-μm-thick patterned SU-2100 photoresist (MicroChem) in order to obtain a single polydimethylsiloxane (PDMS) mold with multiple independent channels. The PDMS mold was punched and sealed on a 75×25 mm microscope glass slide (Thermo Scientific) by plasma treatment (Harrick). Channels were rinsed with isopropanol and distilled water to check proper flow before autoclaving. The height of each channel is 200 μm and the area is 27 mm². Each channel holds, considering the inlet, the outlet and the culture channel itself, 10 μl of medium (FIG. 2).

### Reprogramming

Naïve human iPSCs (niPSCs) or primed iPSCs were generated via mmRNA mediated reprogramming of somatic cells using the described microfluidic platform.

Microfluidic channels were coated with 25 μg/ml fibronectin (Sigma Aldrich) for 30 min at room temperature. Somatic cells were seeded on day −1 of reprogramming at 25 cells/mm² for feeder-free reprogramming in DMEM/10% FBS. Such seeding density allowed robust proliferation and survival of somatic cells during reprogramming. On day 0, 2 hours before the first mmRNA transfection, the medium was changed to PRM, a medium optimized from mRNA transfection and previously used for derivation of primed PSCs (StemMACS ReproBREW XF, Miltenyi biotec). When indicated, 1 μM CH, 1 μM CH and 5 μM Ri were added to PRM. Transfection period is of 9 h. Cells were transfected daily at 9 am, and fresh PRM was given daily at 6 pm. On day 6, medium was changed with a naïve supporting medium according to table 1 and the same regime of transfection and fresh medium change was maintained until day 12. From day 0 to 12, media were supplemented with 200 ng/mL⁻¹ B18R (eBioscience). Transfections using a OSKMN+nGFP mmRNA mix (POU5F1, SOX2, KLF4, c-MYC, NANOG, nuclear GFP) were started on day 0 and daily repeated for 12 d. A mix without nGFP mmRNA was used from day 5 to perform any staining at the end of reprogramming. An incremental dosage of mmRNAs during the first three daily transfections was used, with 50%, 75%, 100% mmRNA amount of subsequent transfections at 0.28 ng/mm². The transfection mix was prepared according to the StemMACS mRNA transfection kit (Miltenyi biotec): 100 ng/μl mmRNA mix of OSKMNG, with stoichiometry 3:1:1:1:1:1, was diluted in transfection buffer (TB) by mixing 10 μl of mmRNA mix with 30 μl of TB. Transfection reagent (TR) was diluted separately in TB by mixing 3 μl of TR with 37 μl of TB. The two solutions were mixed (final volume=80 μl) and incubated for 20 min. For each microfluidic channel, 1.2 μl of transfection solution were diluted in 8.8 μl of medium (either PRM or naïve medium) and added to the cells (corresponding to 0.28 ng mm⁻²).

Reprogramming was performed in hypoxia if not stated otherwise. Reprogramming of fibroblasts towards primed-state was achieved using the protocol described above, keeping PRM until day 12. Importantly, to allow a direct comparison of the reprogramming trajectories towards primed and naïve pluripotency, the same seeding density of 25 cell mm⁻² and the same OSKMN mmRNA cocktail was used.

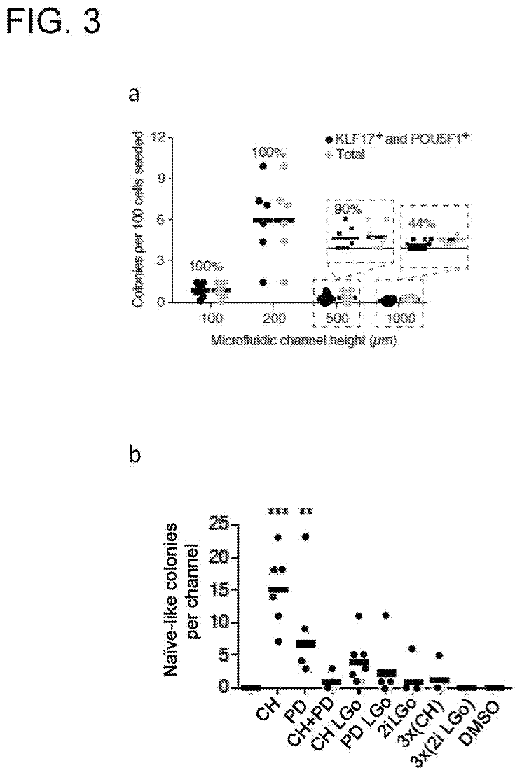

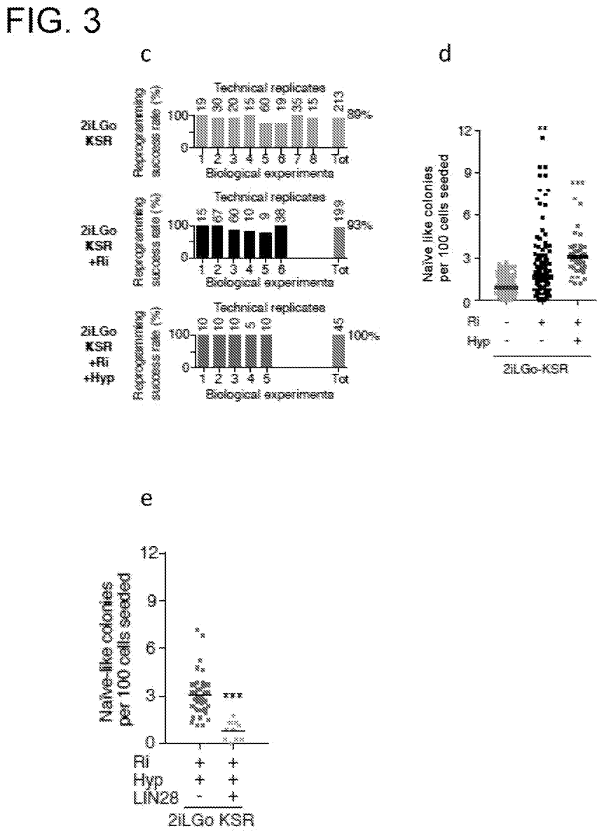

When the effect of different microfluidics channel heights was tested (FIG. 3), the same mmRNA amount per cell was daily delivered, independently of the microfluidic configuration (100 to 1000 μm culture channel heights). For each configuration, mmRNA dosage was progressively increased as in standard protocol (200 μm height) to accommodate cell proliferation.

### In Vitro Differentiation

For germ layer differentiation, niPSCs were seeded on 1% Matrigel coated coverslips with 100 MEF mm⁻² and cultured in RSeT medium for 2 days. Germ layer specific media were used thereafter with daily medium changes; ectoderm medium, for 3 days: KODMEM (Thermo Scientific), 15% KSR, 1% non-essential aminoacids (NEAA), 1% L-glutamine, 0.1 μM LDN193189 (Miltenyi biotec) and 20 ng ml⁻¹ hFGF2 (Peprotech); for the following 9 days hFGF2 was replaced with 10 μM SB431542 (Miltenyi biotec); mesoderm medium, for 6 days: RPMI with 2% B27 (Thermo Scientific), 20 ng ml⁻¹ hFGF2 (Peprotech), 50 ng ml⁻¹ hBMP4 (R&D), 3 μM CH (only first 2 d); endoderm medium, for 6 days: RPMI with 2% B27, 100 ng ml⁻¹ Activin-A (Peprotech), 3 μM CH (only first 2 d). Embryoid bodies (EB): niPSC colonies were mechanically scratched with a tip and transferred in ultra-low adhesive wells (Corning) in the presence of DMEM, 20% FBS, 200 mM L-glutamine, 1% NEAA, 0.1 mM 2-mercaptoethanol. Medium was changed every other day for 15 days before plating EBs on 1% Matrigel-coated glass plates (Labtek). After 5 days, adherent and spread cells were fixed for immunostaining.

### Neuronal Differentiation

Neuronal differentiation protocol was adapted from Errichelli, L. et al. 2017 FUS affects circular RNA expression in murine embryonic stem cell derived motor neurons. Nat. Commun. 8, 14741. Briefly, niPSCs were seeded as single cells at high density (530 cells mm⁻²) on Matrigel-coated plates. Cells were cultured 2 days in RSeT. On day 0, cells were cultured in N2B27, 1% NEAA, 200 ng ml⁻¹ L Ascorbic Acid (Neural Medium, NM) supplemented with 20 ng ml⁻¹ bFGF and 0.1 μM LDN193189. Medium was refreshed daily. On day 3, medium was changed to NM supplemented with 0.1 μM LDN193189 and 10 μM SB431542. On days 4-9, the same medium was supplemented with 1 μM all-trans Retinoic Acid (RA, Sigma Aldrich) and 1 μM SAG (Calbiochem) and was refreshed daily. On days 10-15, medium was changed to NM supplemented with 5 μM DAPT (Sigma Aldrich) and 4 μM SU-5402 (Sigma Aldrich), 1 μM RA and 1 μM SAG, and was refreshed daily. On day 16, cells were dissociated with TrypLE for 10 minutes; 2 volumes of NM were added to inhibit dissociation. Cells were seeded on Matrigel-coated glass coverslips in well and cultured in maturation medium based on NM supplemented with 20 ng ml⁻¹ BDNF, 10 ng ml⁻¹ GDNF, 10 ng ml⁻¹ CNTF (PeproTech), and 10 μM ROCK inhibitor for the first 24 hours. Fresh maturation medium was provided daily up to day 22 when cells were fixed for immunostaining.

### Hepatic Differentiation

Hepatic differentiation protocol was adapted from Hay, D. C. et al. 2008 Efficient differentiation of hepatocytes from human embryonic stem cells exhibiting markers recapitulating liver development in vivo. Stem Cells Dayt. Ohio 26, 894-902 and Hay, D. C. et al. 2008 Highly efficient differentiation of hESCs to functional hepatic endoderm requires ActivinA and Wnt3a signaling. Proc. Natl. Acad. Sci. U.S.A 105, 12301-12306. Briefly, niPSCs were seeded as single cells (20 cells mm⁻²) on Matrigel-coated plates with sparse MEF (50 cell mm⁻²). Cells were cultured 2 days in RSeT and medium. On day 0, cells were cultured in RPMI, 2% B27, 3 μM CH. Same medium with the addition of 100 ng ml⁻¹ Activin-A was refreshed on day 1-2. On day 3-8, medium was changed to KO-DMEM with 20% KSR, 2 mM L-glutamine, 1% NEAA, 0.1 mM 2-mercaptoethanol, 1% DMSO, and changed every other day. On day 9-15, cells were cultured in maturation medium based on L15 basal medium, 8% FBS, 8% tryptose phosphate broth (Sigma), 10 μM hydrocortisone (Sigma), 1 μM insulin, 2 mM L-glutamine, 10 ng mL-1 HGF (Peprotech), 20 ng ml⁻¹ Oncostatin-M (Peprotech). Fresh maturation medium was provided every other day.

### Immunofluorescence and Stainings

Immunofluorescence analysis was performed either on 1% Matrigel-coated glass coverslips in wells or in situ in microfluidic channels with the same protocol. Cells were fixed in 4% (w/v) paraformaldehyde (Sigma-Aldrich) in PBS for 10 min and blocked in 58 horse serum with 0.3% (v/v) Triton-X-100 (Sigma-Aldrich) for 1 h. Blocking buffer: PBS 1:2 was used to dilute primary antibodies. The primary antibodies used are listed in Table 2.

TABLE 2

| Antibody | Dilution | Product code | Company |
|---|---|---|---|
| POU5F1 | 1:250 | sc-5279 | Santa Cruz |
| KLF4 | 1:250 | sc-20691 | |
| SSEA-4 | 1:250 | sc-21704 | |
| CYP3A | 1:200 | sc-365415 | |
| HNF4A | 1:200 | sc-6556 | |
| NANOG | 1:100 | RCAB0004P-F | Reprocell |
| DPPA3 | 1:200 | MAB4388 | Millipore |
| KLF17 | 1:250 | HPA024629 | Atlas |
| TFE3 | 1:250 | HPA023881 | |
| TUJ1 | 1:1000 | MMS-435P | Covance |
| DNMT3L | 1:100 | A-1005-050 | Epigentek |
| TFCP2L1 | 1:250 | AF5726 | R&D |
| SOX1 | 1:100 | AF3369 | |
| GATA4 | 1:150 | AF2606 | |
| SOX17 | 1:200 | AF1924 | |
| NESTIN | 1:150 | MAB1259 | |
| GATA6 | 1:150 | AF1700 | |
| OTX2 | 1:100 | AF1976 | |
| FOXA2 | 1:400 | D56D6 | Cell signaling |
| SOX2 | 1:250 | NB110-37235 | Novus |
| AFP | 1:200 | A8452 | Sigma-Aldrich |
| VIMENTIN | 1:200 | SAB1305445 | |
| T | 1:100 | ab20680 | Abcam |
| MAP2 | 1:1000 | ab32454 | |
| NEUN | 1:100 | ab177487 | |
| H3K9me3 | 1:500 | ab8898 | |
| 5-mC | 1:250 | BI-MECY-0100 | Eurogentec |

For staining with 5-methylcytosine (5mC), fixed samples were permeabilized with 0.5% Triton X-100 for 1 h and treated with 2 N HCl for 30 min at room temperature to denature DNA. Samples were neutralized with PBS before blocking and antibody incubation. Alexa488, Alexa568, or Alexa647-conjugated anti-rabbit, anti-mouse, or antigoat secondary antibodies raised in donkey (A-21206, A10042, A-21202, A-31571, A10037, A-11057, A-11055, Thermo Scientific) were incubated at room temperature for 45 min at 1:500.

Nuclei were stained with either Hoechst 33342 (Thermo Scientific) or DAPI (Sigma-Aldrich).

For mitochondrial staining, cells were incubated with 1:50000 TMRM and 1:20000 MitoTracker (T668, M7514, Thermo Scientific) for 30 min in culture medium at 37° C. and washed twice in PBS before image acquisition.

For EdU staining, cells were exposed to an EdU pulse of 1 h before fixation in formaldehyde for 15 min. Samples were processed with Click-iT EdU Alexa Fluor 488 Imaging kit and counterstained with Hoechst nuclear dye (all Thermo Scientific). Fluorescence images were acquired through a Leica SP5 II confocal system or a Leica 6000B epifluorescence microscope.

For AP staining, cells were fixed with a citrate-acetone-formaldehyde solution and stained using an alkaline phosphatase kit (Sigma-Aldrich). Plates were scanned using an Epson scanner and scored manually.

### Flow Cytometry and Cell Sorting

Single cells in suspension were obtained by incubating samples for 5 min with TrypLE. For DNA-content analysis by flow cytometry, cells pellets fixed in cold 70% ethanol were resuspended in PBS, incubated with 50 μg ml⁻¹ propidium iodide, and processed with FACSCanto™ II (BD). For cell sorting based on DNA-content, cells were resuspended in naïve medium with 10 μM Ri and 5 μM Vybrant DyeCycle Ruby live stain (Thermo Scientific) for 30 min before sorting. Sorted samples were cultured on new MEFs with 10 μM Ri for 24 h.

### Karyotype

Cells were incubated with 0.06 μg ml⁻¹ KaryoMAX (Thermo Scientific) in culture medium for 6 h at 37° C. niPSCs were isolated with ReLeSR (Stemcell technologies) and resuspended in PBS before centrifugation. Pellet was resuspended in pre-warmed 75 mM KCl for 10 min at 37ºC. After centrifugation, pellet was gently resuspended in 1 ml of freshly-prepared fixative (3:1 methanol:acetic acid). This step was repeated twice. Q-banded fixed samples were analysed by Research & Innovation S.p.A. (Padova, Italy).

### Quantitative PCR

Total RNA was isolated using Total RNA Purification Kit (Norgen Biotek), and complementary DNA (CDNA) was made from 500 using ng M-MLV Reverse Transcriptase (Invitrogen) and dN6 primers. For real-time PCR SYBR Green Master mix (Bioline. Cat. BIO-94020) was used. Primers are detailed in Table 3. Three technical replicates were carried out for all quantitative PCR. GAPDH was used as endogenous control to normalize expression.

TABLE 3
Forward and reverse primers (5′>3′)

| Gene | Forward | Reverse |
|---|---|---|
| GAPDH | CGAGATCCCTCCAAAAT | GGCAGAGATGATGACCC |
| | CAA | TTT |
| | (SEQ ID no. 1) | (SEQ ID no. 2) |
| NANOG | TTTGTGGGCCTGAAGAA | AGGGCTGTCCTGAATAA |
| | AACT | GCAG |
| | (SEQ ID no. 3) | (SEQ ID no. 4) |
| OCT4 | GTGGAGGAAGCTGACAA | ATTCTCCAGGTTGCCTC |
| | CAA | TCA |
| | (SEQ ID no. 5) | (SEQ ID no. 6) |
| SOX2 | CCAGCAGACTTCACATG | ACATGTGTGAGAGGGGC |
| | TCC | AGT |
| | (SEQ ID no. 7) | (SEQ ID no. 8) |
| PRDM14 | GAGCCTTCAGGTCACAG | TCCACACAGGGGGTGTA |
| | AGC | CTT |
| | (SEQ ID no. 9) | (SEQ ID no. 10) |
| DPPA5 | AAGTGGATGCTCCAGTC | ATCCAAGGGCCTAGTTC |
| | CAT | GAG |
| | (SEQ ID no. 11) | (SEQ ID no. 12) |
| TFCP2L1 | GGAGTTCCAGCCATGCT | CCTGCTTGAAGATGGGC |
| | CTT | AGA |
| | (SEQ ID no. 13) | (SEQ ID no. 14) |
| KLF4 | CCCAATTACCCATCCTT | CAGGTGTGCCTTGAGAT |
| | CCT | GG |
| | (SEQ ID no. 15) | (SEQ ID no. 16) |
| OTX2 | CAAAGTGAGACCTGCCA | TGGGACAAGGGAATCTG |
| | AAAAGA | ACAGTG |
| | (SEQ ID no. 17) | (SEQ ID no. 18) |
| ZIC2 | CATGCACGGTCCACACC | CTCATGGACCTTCATGT |
| | TC | GCTT |
| | (SEQ ID no. 19) | (SEQ ID no. 20) |
| MT-ND1 | CGATTCCGCTACGACCA | GTTTGAGGGGGAATGCT |
| | ACT | GGA |
| | (SEQ ID no. 21) | (SEQ ID no. 22) |
| MT-ND4 | TTCCTCCGACCCCCTAA | GATAAGTGGCGTTGGCT |
| | CAA | TGC |
| | (SEQ ID no. 23) | (SEQ ID no. 24) |
| MT-ND4L | TCTCATAACCCTCAACA | AGGCGGCAAAGACTAGT |
| | CCCAC | ATGG |
| | (SEQ ID no. 25) | (SEQ ID no. 26) |
| LIN28A | CTGTAAGTGGTTCAACG | CCATGTGCAGCTTACTC |
| | TGCG | TGGT |
| | (SEQ ID no. 27) | (SEQ ID no. 28) |
| ECAD | CCCACCACGTACAAGGG | CTGGGGTATTGGGGGCA |
| | TC | TC |
| | (SEQ ID no. 29) | (SEQ ID no. 30) |
| DUSP6 | AACAGGGTTCCAGCACA | GGCCAGACACATTCCAG |
| | GCAG | CAA |
| | (SEQ ID no. 31) | (SEQ ID no. 32) |
| SPRY4 | GGGAGCCACTGAGAACA | TGGCTCCTAAATCCATC |
| | GAG | CTG |
| | (SEQ ID no. 33) | (SEQ ID no. 34) |
| SOX17 | ACGCCGAGTTGAGCAAG | TCTGCCTCCTCCACGAA |
| | A | G |
| | (SEQ ID no. 35) | (SEQ ID no. 36) |
| GATA6 | GCAAAAATACTTCCCCC | TCTCCCGCACCAGTCAT |
| | ACA | C |
| | (SEQ ID no. 37) | (SEQ ID no. 38) |
| AFP | GTGCCAAGCTCAGGGTG | TCCAACAGGCCTGAGAA |
| | TAG | ATC |
| | (SEQ ID no. 39) | (SEQ ID no. 40) |
| MESP1 | CTGTTGGAGACCTGGAT | CGTCAGTTGTCCCTTGT |
| | GC | CAC |
| | (SEQ ID no. 41) | (SEQ ID no. 42) |
| ACTA2 | GGAAAAGATCTGGCACC | GAGTCATTTTCTCCCGG |
| | ACT | TTG |
| | (SEQ ID no. 43) | (SEQ ID no. 44) |
| NEUN | CCACCATTTTCCCAGGT | ATTTTCCCCGAGGCACT |
| | CT | CT |
| | (SEQ ID no. 45) | (SEQ ID no. 46) |
| TUBB3 | TTTCATCTTTGGTCAGA | TCGCAGTTTTCACACTC |
| | GTGG | CTTC |
| | (SEQ ID no. 47) | (SEQ ID no. 48) |
| NCAD | CGTGGTCAAACCAATCG | AACAGACACGGTTGCAG |
| | AC | TTG |
| | (SEQ ID no. 49) | (SEQ ID no. 50) |
| SOX1 | GCTGACACCAGACTTGG | CCCCTCGAGCAAAGAAA |
| | GTTT | ACG |
| | (SEQ ID no. 51) | (SEQ ID no. 52) |
| TNNT2 | GACAGAGCGGAAAAGTG | CCTTCTCCCTCAGCTGA |
| | GGAA | TCTT |
| | (SEQ ID no. 53) | (SEQ ID no. 54) |
| NKX2.5 | ACCTCAACAGCTCCCTG | ATAATCGCCGCCACAAA |
| | ACTC | CTCTCC |
| | (SEQ ID no. 55) | (SEQ ID no. 56) |
| FLK1/ | GGCGGCACGAAATATCC | GGAGGCGAGCATCTCCT |
| KDR | TCT | TTT |
| | (SEQ ID no. 57) | (SEQ ID no. 58) |
| ACAN | CCCCTGCTATTTCATCG | GACACACGGCTCCACTT |
| | ACCC | GAT |
| | (SEQ ID no. 59) | (SEQ ID no. 60) |
| NES | AGGAGAAACAGGGCCTA | GGAGGGTCCTGTACGTG |
| | CAGA | GC |
| | (SEQ ID no. 61) | (SEQ ID no. 62) |
| GFAP | AGGAGGAGGTTCGGGAA | TCATACTGCGTGCGGAT |
| | CTC | CTC |
| | (SEQ ID no. 63) | (SEQ ID no. 64) |
| NKX6-1 | CACGAGACCCACTTTTT | ACCAGACCTTGACCTGA |
| | CCG | CTCT |
| | (SEQ ID no. 65) | (SEQ ID no. 66) |
| VIM | CTCCACGAAGAGGAAAT | GTGAGGTCAGGCTTGGA |
| | CCA | AAC |
| | (SEQ ID no. 67) | (SEQ ID no. 68) |
| EPCAM | TGGACATAGCTGATGTG | CCAGGATCCAGATCCAG |
| | GCTTA | TTG |
| | (SEQ ID no. 69) | (SEQ ID no. 70) |
| ICAM | CAAGGCCTCAGTCAGTG | CCGGAAAGCTGTAGATG |
| | TGA | GTC |
| | (SEQ ID no. 71) | (SEQ ID no. 72) |
| ALB | ACCCCACACGCCTTTGG | CACACCCCTGGAATAAG |
| | CACAA | CCGAGCT |
| | (SEQ ID no. 73) | (SEQ ID no. 74) |
| CCNA2 | TTTGATAGATGCTGACC | ATGCTGTGGTGCTTTGA |
| | CATACC | GGT |
| | (SEQ ID no. 75) | (SEQ ID no. 76) |
| IL6 | CCAGAGCTGTGCAGATG | GCATTTGTGGTTGGGTC |
| | AGT | AGG |
| | (SEQ ID no. 77) | (SEQ ID no. 78) |

### RNA-Seq

niPSCs were preferentially isolated from mEFs with ReLeSR. Briefly, cells were washed with PBS, incubated with ReLeSR for 60 s, and left with a film of liquid for 7 min. N2B27 medium was added and pipetted 4-5 times to detach colonies. Total RNA was isolated as above and sequenced with an Illumina NexSeq500, in 75 bp pair-end prepared following standard format. Libraries were protocols from Illumina using TruSeq Stranded mRNA Library Kit. A total of ˜249M reads were produced from the 13 samples (3 fibroblasts, 1 primed hiPSC and 9 naïve cells lines; 19M reads per sample on average). Expression levels for all the genes from ENSEMBL 87 were quantified using RSEM 1.3.053 with STAR 2.5.2b54 (human genome GRCh38.p7). The genome index for STAR alignment was prepared using “rsem-prepare-reference” with options ‘--starsjdboverhang’ set to mean read length minus 1 according to STAR guidelines. Alignment and quantification were performed with default parameters using stranded pair-end mode. Gene expression level quantification of 21 samples collected from public available datasets has been done as described for the in-house samples. The RSEM parameters were set according to the library design (mean reads length, stranded/non-stranded, paired/single end, see Table 4).

TABLE 4
List of sample accession from GEO/ENA databases
with read average length, library layout (single-
end or pair-end) and library strategy

| Sample | Average read | Library | Library |
|---|---|---|---|
| Accessions | length: | layout | strategy |
| GSM2448850 | 50 | Single | U |
| GSM2448851 | 50 | Single | U |
| GSM2448852 | 50 | Single | U |
| GSM1553088 | 50 | Single | U |
| GSM1553089 | 50 | Single | U |
| GSM1553090 | 50 | Single | U |
| ERS537878 | 101 | Paired | U |
| ERS537890 | 101 | Paired | U |
| ERS559336 | 75 | Paired | U |
| ERS559335 | 75 | Paired | U |
| ERS559332 | 75 | Paired | U |
| ERS559334 | 75 | Paired | U |
| GSM2218660 | 101 | Paired | S |
| ERS537884 | 101 | Paired | U |
| GSM2218670 | 101 | Paired | S |
| GSM2218671 | 101 | Paired | S |
| ERS1059993 | 125 | Paired | U |
| ERS1059996 | 125 | Paired | U |
| ERS1059999 | 125 | Paired | U |
| GSM2218668 | 101 | Paired | S |
| GSM2218669 | 101 | Paired | S |

(U = Unstranded; S = Stranded).

The final expression matrix has been generated by excluding those genes that did not have more than 10 raw counts in at least 3 out of 34 samples. After this 5 filter, the expression of 20936 genes was obtained. All RNA-seq statistical analyses were carried out in R environment (version 3.4.3) with Bioconductor 3.6. Differentially expressed genes (DEG) were computed among the 3 groups (primed, naïve, fibroblast) using edgeR 55 (function call “fit=glmQLFit (counts, design=˜ group); glmTreat (fit, coef=n, lfc=2)” where ‘n’ is one of the contrasts). A gene was considered a DEG when the absolute value of log 2 fold change was higher than 2 and the adjusted p-value was smaller than 0.05 (p.adjust function, Benjamini-Hochberg method). Principal component analysis (PCA) was performed using log 2-normalized pseudo-counts (defined as count plus 1) with prcomp function with default parameters using DEGs. Heatmaps were made using the log 2-normalized pseudo-counts (unless stated otherwise) with pheatmap function from pheatmap R package (version 1.0.8, distance used ‘euclidean’, ‘ward’ linkage, scale=′ row′) on DEGs or selected markers. The raw counts were normalized using betweenLaneNormalization function with upper quantile method (EDAseq R package).

### Transposon Analysis

Transposons coordinates were downloaded from UCSC repeat masker track (hg38). Transposons overlappping any genomic features annotated in ENSMBL87 were filtered out. The raw reads were aligned using bowtie2 sensitive and end-to-end mode on the human genome GRCh38.p7. Transposon expression was quantified using bamtools multicov (v2.26.0). Transposons with at least 20 cpm (count per million) in at least one sample were analysed. Differentially expressed transposons were identified using edgeR R package. A transposon was considered differentially expressed when the p-value was lower or equal to 0.05 and the log 2 fold change greater or equal to 2. Heatmap was made using the log 2-normalized pseudo-counts scaled by row means with pheatmap R package as describe for RNA-seq data.

### SNP Analysis for Imprinting and X Re-Activation

Reads with a minimum quality of 30 (samtools view -q 30 align.bam align-f.bam) were extracted from the alignments. Using GATK haplotypeCaller (genotyping_mode: DISCOVERY, minReadsPerAlignmentStart: 5, max_alternate_alleles: 1, stand_conf_call: 1 and filter_reads_with_N_cigar) allele counts over the SNPs of dbSNP human version b149 have been generated. Non biallelic variants as well as In-DELs were filtered out. Moreover, only SNPs with at least 10 reads in at least one sample were taken into consideration.

A SNP was considered heterozygous when the ratio of the counts between minor and major alleles was greater than 0.2 and the minor allele has at least 5 reads. Minor allele is the allele with fewer counts.

The loss of imprinting (LOI) of a gene was quantify as the number of SNP that shows heterozygosity in each gene. A LOI was defined when at least 2 SNPS show heterozygosity in a gene. LOI were tested on those genes associated to the imprinted regions defined for the methylation analysis.

X re-activation (Xa) was quantify selecting the heterozygous SNP from X-chromosome genes and by plotting the minor/total allele count ratios of heterozygous SNP (defined as above). The pseudo-autosomals regions (PAR1: chrX: 10,000-2,781,479 and chrY: 10,000-2,781,479; PAR2: chrX: 155,701,383-156,030,895 and chrY: 56,887,902-57,217,415) were excluded from the analysis.

### Methylation Analysis

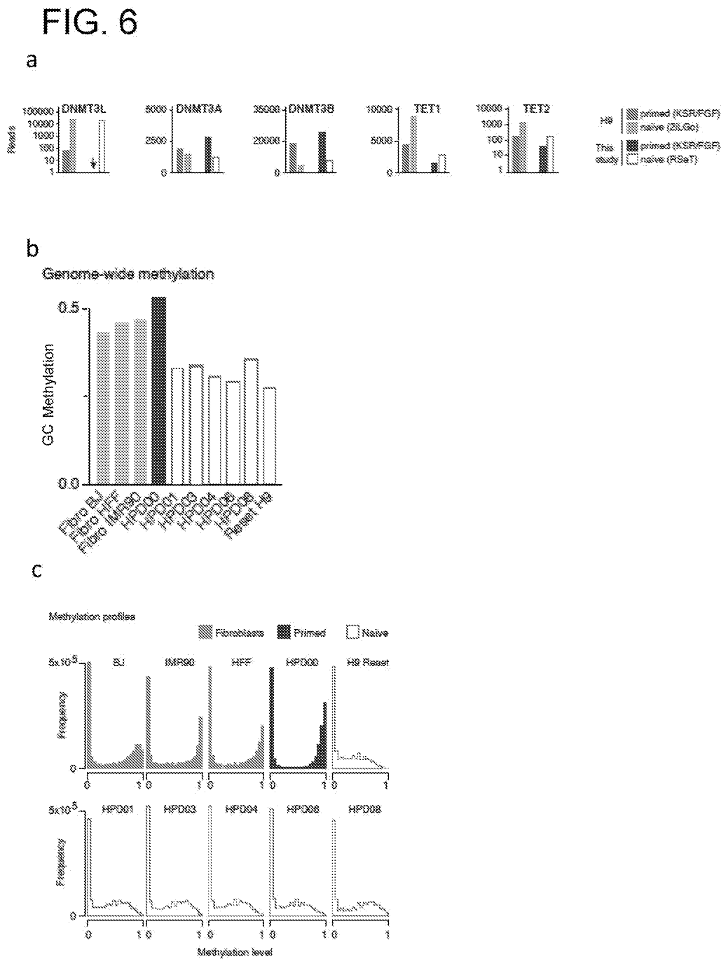

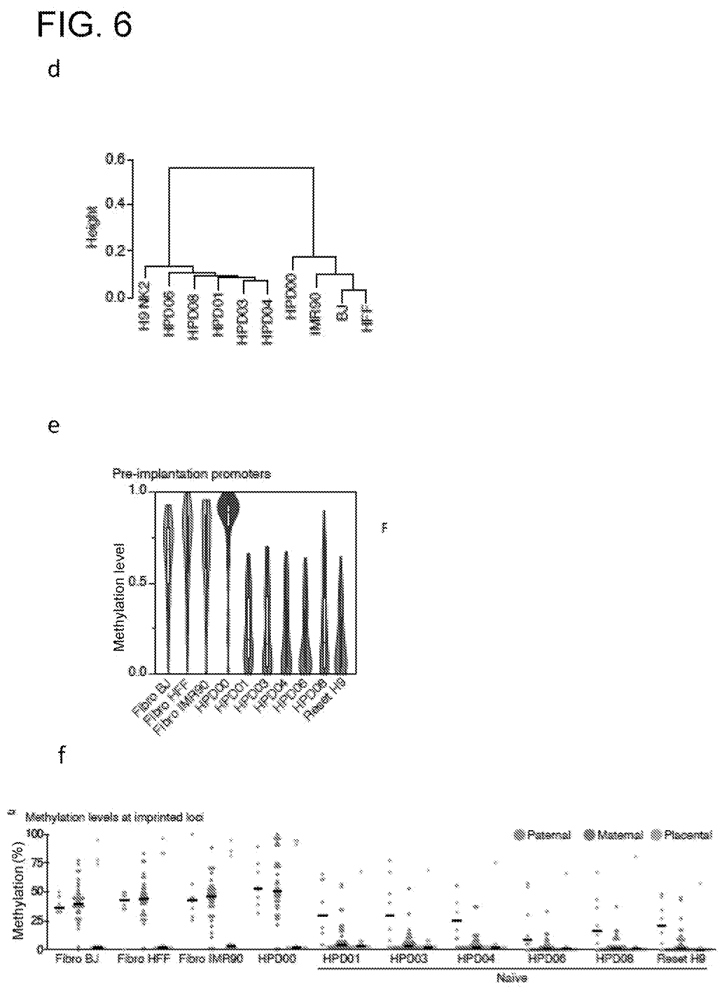

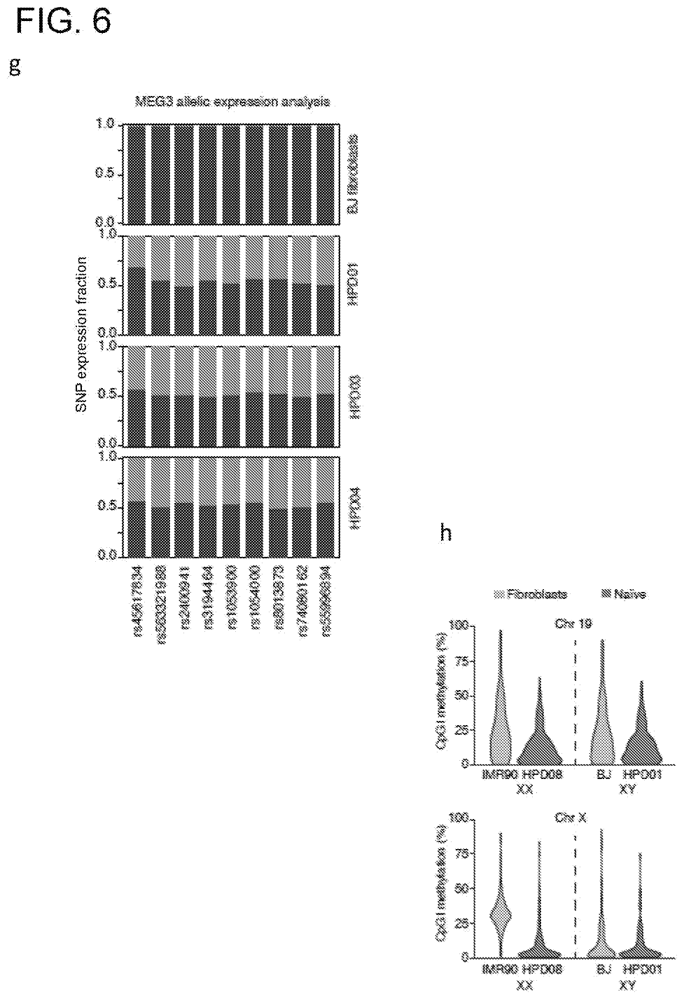

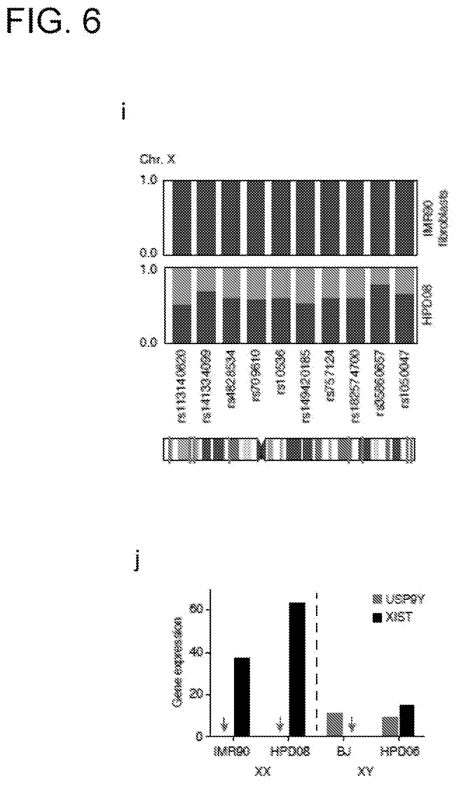

RRBS libraries were produced using the Ovation RRBS Methyl-Seq System (NuGEN) starting from 100 ng of genomic DNA extracted with the Quick-DNA Plus kit (Zymo) according to manufacturer's specifications. Libraries were sequenced on a NextSeq 500 (Illumina) using a single-end 75-cycle high-output flow cell. Sequence reads were first trimmed using Trim Galore software to remove adapter sequences and low-quality end bases and then trimmed with a custom python script provided by NuGEN Technical Support to remove any read that does not contain an MspI site signature (YGG) at the 5′-end. Reads alignment on hg19 reference sequence and methylation calling was then performed with bismark. Coordinates of DMRS were obtained from literature (Cacchiarelli, D. et al. 2015 Integrative Analyses of Human Reprogramming Reveal Dynamic Nature of Induced Pluripotency. Cell 162, 412-424; Okae, H. et al. 2014 Genome-wide analysis of DNA methylation dynamics during early human development. PLOS Genet. 10, e1004868). Subsequently, average DNA methylation levels and total coverage for each DMR regions was determined for all bismarck processed RRBS data files using R and the methylkit package. To that end, RRBS data files were processed with the methRead function and the bismarck Coverage parameter set. Next, the regionCounts function was used to determine the number of methylated and unmethylated C's in each of the DMRs in each sample. These values were then combined using the unite( ) function with the min.per.group parameter set to 1. Finally, only those regions that were covered by at least 5 reads and computed a coverage weighted average methylation level for each region across all CpGs that were covered were retained. These values were then plotted in FIG. 6 e, f.

### Image Analysis

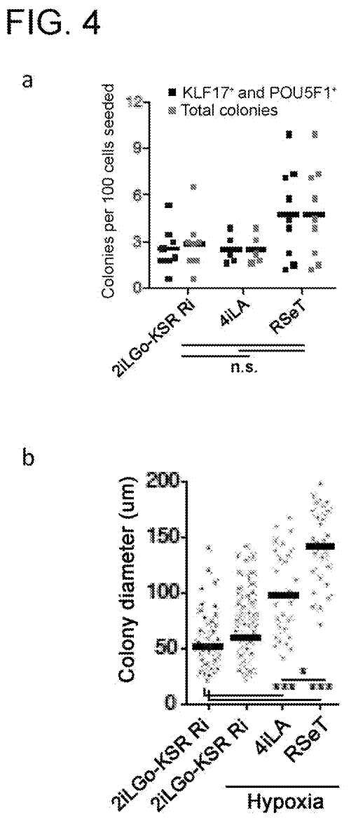

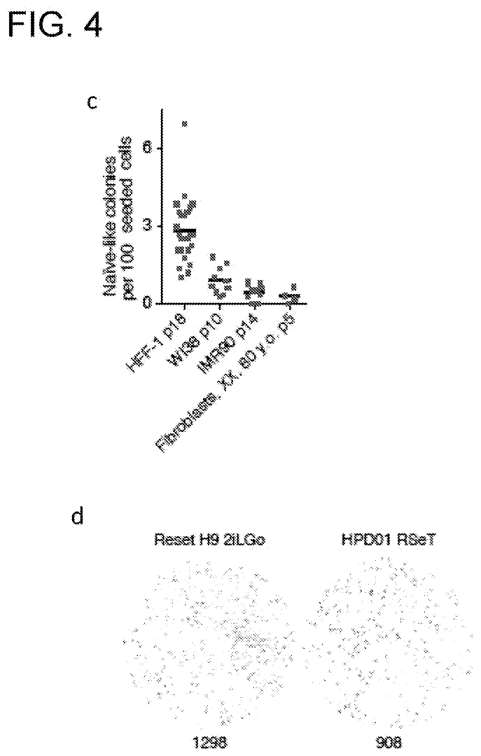

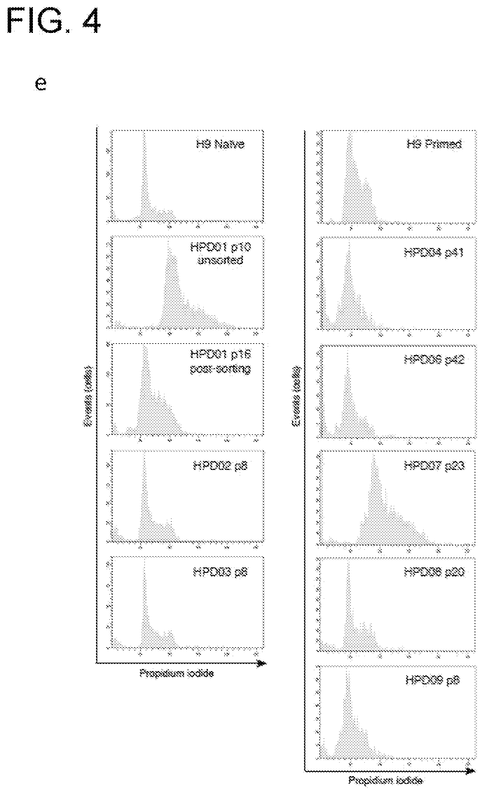

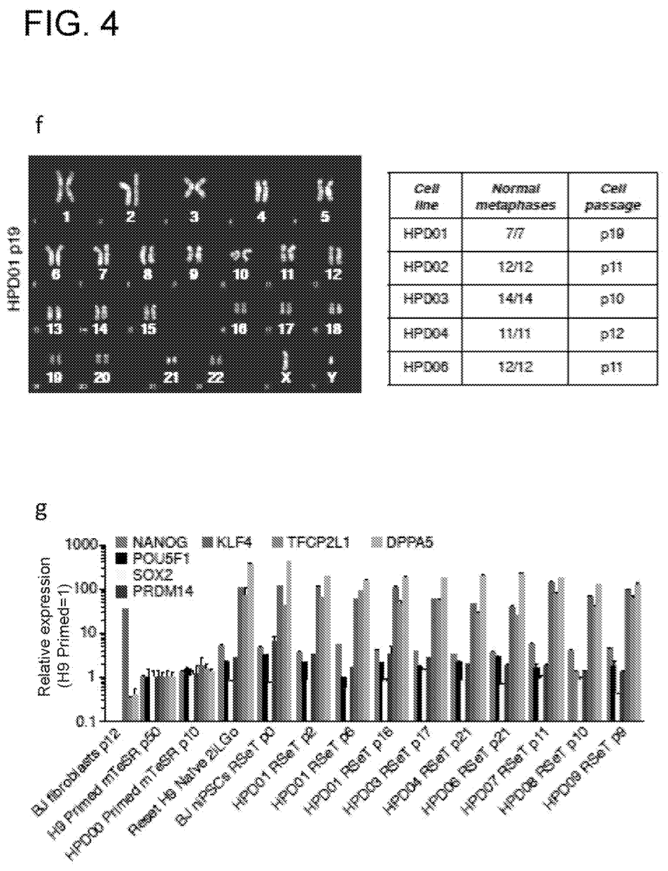

Fiji 1.0 (ImageJ2) was used for image analysis. The size of niPSC colonies (FIG. 4b) was measured by delimiting a colony area and calculating the equivalent diameter as (area/π)\hat{ }0.5 (Shape filter plugin function). Fluorescence intensity (H3K9me3 or 5mC) across nuclei was measured with the Plot Profile function. For each cell line representative fields were selected and 6 nuclei were randomly chosen, as in Takashima et al. 2014 (cited).

### Statistical

For statistical analyses, multiple comparisons were performed by one-way ANOVA with Tukey post-hoc test. Single pairwise comparisons were analysed using Student's t test. Normality assumption was verified with D'Agostino-Pearson normality test in Prism (Graphpad). Kruskal-Wallis test was used with non-normal datasets. P-values were indicated as follows: P<0.05 (*), P<0.01 (**) or P<0.001 (**), not significant (n.s.). Values are expressed by means and standard deviation (s.d.) throughout the text, n indicates the number of replicates, referring to a combination of independent experiments performed in at least two independent experiments. For reprogramming experiments, a minimal sample size of 2 biological with 2 technical replicates each was used as lab routine. A minimal sample size of 5 technical replicates was adopted for each biological replica in microfluidic experiments.

### Results

Evaluation of Reprogramming Media Allowing Formation of Naïve-Like iPSC Colonies.

Human fibroblasts were plated at day −1 in microfluidic in DMEM+10% FCS. At day 0, media was changed into PRM and transfection with OSKMN started, for 6 days. At day 6, PRM was replaced with 2Li supplemented with 1% knockout serum replacement (KSR) and the PKC inhibitor Gö6983 (2LiGo+KSR). Each column in the graph in FIG. 3b shows the number of colonies for each microfluidics channel as a dot. Mean of each condition is indicated as a bar. n=2 biological experiments. During the first 6 days, the addition of various inhibitors was tested and the formation n of several naïve-like colonies under different combinations was observed, with the most efficient condition being PRM+CH The tested conditions were as follows: PRM only (−), PRM+1 μM CHIR99021 (CH), PRM+1 μM PD0325901 (PD), PRM+1 μM CHIR99021+1 μM PD0325901 (CH+PD), PRM+1 μM CHIR99021+10 ng/ml human LIF+2 μM Gö6983 (CH L Go), PRM+1 μM PD0325901+10 ng/ml human LIF+2 μM Gö6983 (PD L Go), PRM+1 μM PD0325901+1 μM CHIR99021+10 ng/ml human LIF+2 μM Gö6983 (21L Go), PRM+3 μM CHIR99021 (3×CH), PRM+3 μM PD0325901+3 μM CHIR99021+30 ng/ml human LIF+6 μM Gö6983 (3×2iL Go)

Best results in term of formed colonies were observed when using PRM in the presence of the GSK inhibitor CH. Fibroblasts reprogramming using PRM+CH followed by 2iLGo−KSR at day 6 leads to naïve-like colonies at day 12. Fibroblasts convert from a spindle-like (day 1) to an epithelial-like morphology (day 5-6). Cells become progressively compact (day 7) and small colonies emerge from day 9. mmRNA transfection was robust (data not shown), MET occurred by day 5 and was followed by appearance of naïve-like colonies from day 9 (data not shown).

Freshly-derived colonies were then cultured for 3 days without mmRNAs (day 15) to allow their complete clearance, and analyzed in situ for naïve or primed associated markers. Robust protein expression of naïve markers TFCP2L1, DPPA3 and DNMT3L (data not shown). In addition, the colonies expressed the core pluripotency factor NANOG and were negative for the primed surface maker SSEA4. Success rate, measured as the percentage of microfluidic channels containing naïve-like colonies, was evaluated (FIG. 3c). Each bar indicates an independent experiment, the number above each bar indicates the number of microfluidic channels (i.e. technical replicates) used in each experiment. Efficiency of generation of naïve-like colonies calculated as the number of colonies observed at day 12 per 100 cells seeded is showed in FIG. 3d. The number of colonies for each microfluidics channel is indicated as a dot. Mean of each condition is indicated as a bar. The protocol appeared robust given that 89% of each individual μF channel presented naïve-like colonies (FIG. 3c-d, 2LiGo+KSR, n=213 in 8 independent experiments) with an efficiency of 0.87% (i.e., 0.87 colonies generated for 100 fibroblasts seeded). The effect of inhibition of rho-associated protein kinase (ROCK) and hypoxia was then evaluated. 100% μF channels contained colonies, with an increased efficiency of (FIG. 3c-d, 2LiGo+KSR+Ri+Hyp). Both ROCK 3.02% inhibitor and Hypoxia increased colony formation efficiency. Addition of LIN28 mmRNA to the OSKMN cocktail in 2iLGo+KSR+Ri in hypoxia causes a marked reduction in efficiency, in line with the role of LIN28 as a factor promoting primed pluripotency (FIG. 3e). The reported experiments clearly demonstrate that the delivery of mmRNAs for OSKMN in μF allows rapid and robust generation of naïve PSCs cells from human fibroblasts, according to the signalling environment experienced by the cells.

Long-Term Expansion and Characterization of Naïve iPSCs

Cells were transfected with OSKMN in μF under hypoxia, for 6 days in PRM+CH+Ri, followed by 6 days in different naïve media: 2iLGo−KSR+Ri (modified from Takashima et al., 2014), 4iLA and RSeT (Stemcell Technologies). At day 12 immunostaining for the naïve marker KLF17 and for POU5F1/OCT4 was performed and colonies were counted. Each dot represents a technical replicate, bars indicate means. Colonies formed robustly with all media (FIG. 4a). Morphology and size of representative colonies at day 12 of reprogramming in different naïve supporting media (D6-12) was observed and colony size was quantified by measure of the equivalent diameter. Data are reported in FIG. 4b. 4iLA and RSeT allow the formation of colonies with a larger diameter compared to 2iLGo-based formulations. For each condition, at least 30 colonies (dots) from 5 technical replicates were quantified. Means=bars. Among the 3 media, RSeT medium again appeared the most robust so it has been used for all further experiments. Considering that the extracellular matrix is known to affect PSC behaviour, coating with Fibronectin, Matrigel or Laminin was compared. In all 3 conditions KLF17+/POU5F1+ colonies formed with comparable efficiency (data not shown) demonstrating the here described method can be easily adapted to different culture conditions.

To evaluate whether the obtained primary colonies could be expanded for long term, colonies were transferred from μF to CCC on feeders in the respective media in hypoxia. Colonies were successfully expanded over multiple passages. Naïve associated markers such as TFCP2L1, DPPA3, POU5F1, KLF17, DNMT3L, TFE3, NANOG, KLF4, are expressed after extensive culture (p14) with strongly reduced expression of differentiation marker T and the primed pluripotency markers SSEA4, OTX2 and ZIC2. Bright-field images showed numerous and homogenous dome-shaped colonies after prolonged culture, indicating a stable phenotype (data not shown).

Results were replicated starting from 3 other somatic cells: human male foreskin fibroblasts HFF and the female lung fibroblasts WI-38 and IMR-90 and niPSCs were obtained with comparable efficiency (FIG. 4c), colonies showing naïve markers comparable to reprogrammed BJ samples. niPSCs were also obtained, although at lower efficiency, from primary skin fibroblasts of an 80-year-old female healthy donor. All niPSC lines expressed naïve markers and expanded robustly over multiple passages retaining naïve morphology and high clonogenicity. FIG. 4d is indicative of a clonal assay performed by plating 2000 cells in a well of a 12 well plate with feeder cells. HPD01 niPSCs in RSeT display high clonogenic capacity, comparable to established Reset H9 cells in 2iLGo 21, which served as positive control.

niPSCs either freshly derived (p0) or after several passages (up to p21) were then characterized by qPCR. The conversion appears more rapid than conventional reprogramming to primed state, given that primary colonies start to emerge at day 9 and a naïve transcriptome is acquired within day 15. FIG. 4g reports gene-expression analysis by qPCR of niPSCs showing expression profiles distinct from primed PSCs and comparable to established human naïve Reset H9 cells. Expression relative to Primed H9 ES cells was calculated. GAPDH served as loading control. DNA content was assessed by propidium iodide and cytofluorimetric analysis. Only HPD01 (p10) and HPD07 (p23) showed a preponderant tetraploid population after several passages in CCC and RSeT. Six passages after sorting (p16), HPD01 showed a diploid-like profile and was used thereafter for other characterizations, followed by flow cytometry. It has been observed that for the first 8 passages all lines were diploid. Stable diploid karyotype was observed in 6 out of 8 lines for up to 42 passages. (FIG. 4e). By Fluorescence Activated Cell Sorting (FACS) the diploid population was isolated and cells then maintained a correct karyotype. Correct karyotype has also been confirmed by Q-banding in 5 independent niPSC lines (FIG. 4f), therefore concluding that the process of direct reprogramming in microfluidics does not induce chromosomal abnormalities per se, which are likely due to extended culture.

### Naïve State Transcriptome

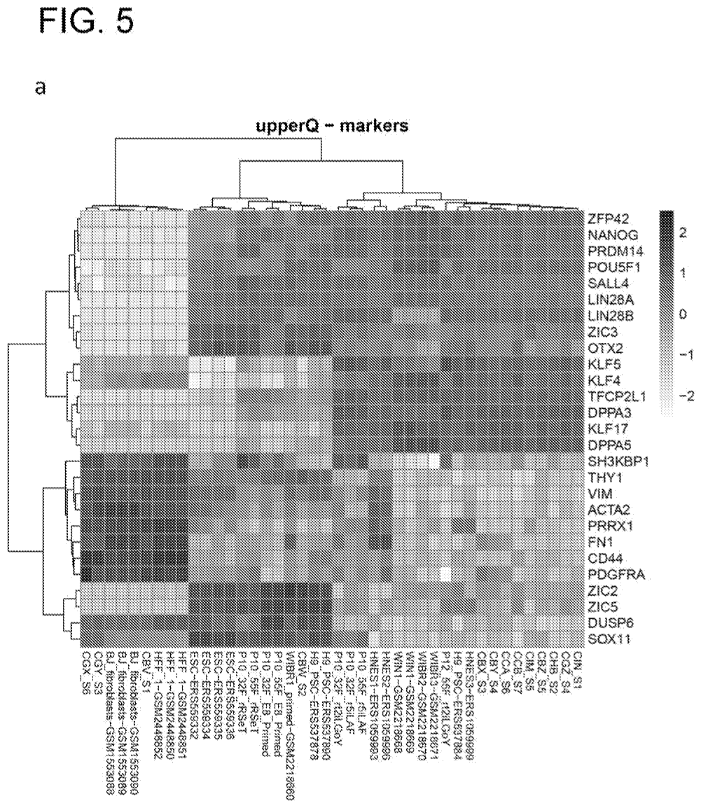

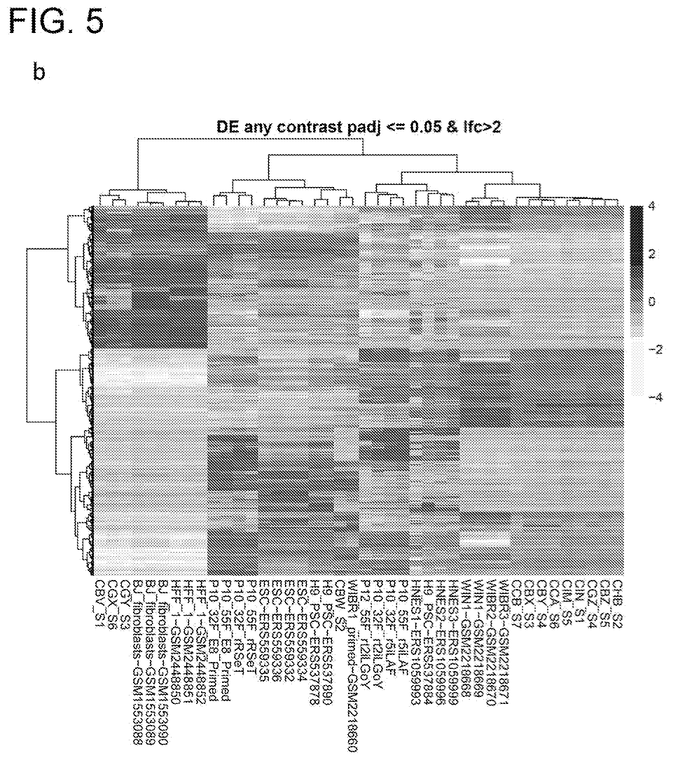

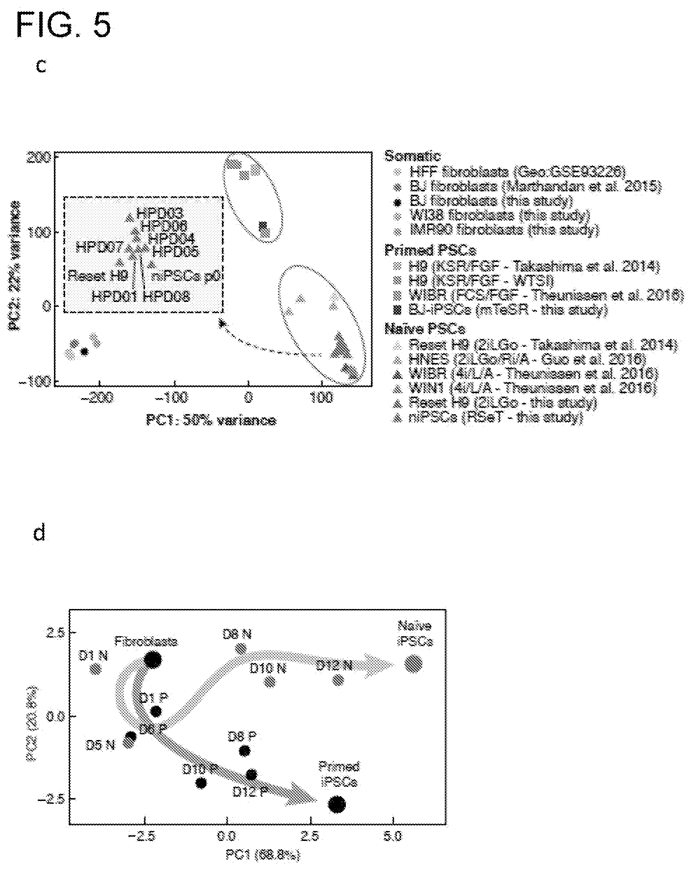

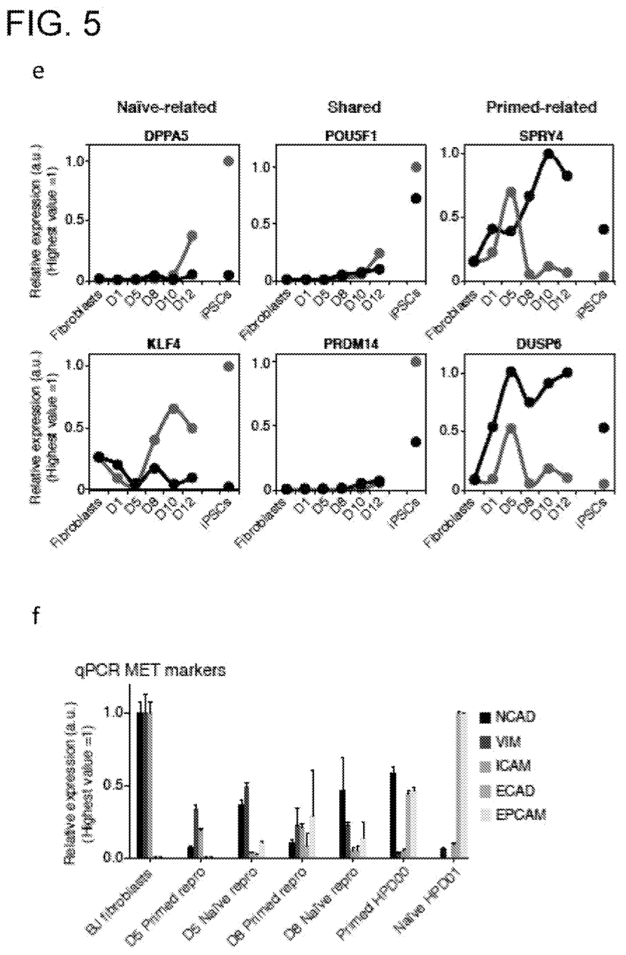

Unsupervised clustering based on markers highly expressed in human naïve ESCs and human embryos, (e.g. TFCP2L1, KLF17, DPPA5 and KLF4) or specifically expressed in primed PSCs (e.g. ZIC2, ZIC5, SOX11) or in fibroblasts (e.g. VIM, FN1 and CD44) were performed. This analysis grouped niPSCs together with other previously described human naïve cells, and clearly separated them from primed PSCs. FIG. 5a is the heatmap of unsupervised hierarchical clustering based on somatic and pluripotency associated markers. Values displayed correspond to the gene expression level (normalized log 2 pseudo-counts) scaled by the row mean. Same results were then obtained by analyzing the whole transcriptome (FIG. 5b). In FIG. 5c is reported a comparison in term of morphology and size of representative colonies at day 12 of reprogramming in different naïve supporting media (D6-12).

The presence of a unique set of transposable elements (TE) expressed by cleavage-stage embryos was used to define the human naïve pluripotent state. The TE profile of niPSCs was analyzed and it was clearly distinct from primed cells and comparable to previously described naïve PSCs (FIG. 5b). Notably, primary colonies (“niPSCs p0” in FIG. 5a-c) collected at day 15 were transcriptionally indistinguishable from established naïve PSCs, indicating the here described reprogramming method allows rapid and full acquisition of a human naïve transcriptome.

The reprogramming toward the naïve and primed states were compared, by transfecting fibroblasts with mmRNAs for OSKMN either under the method according to the present invention to generate niPSCs, or in PRM to generate primed iPSCs. Samples were collected at Day 1, 5, 8, 10 and 12 and the expression of pluripotency markers was analyzed. PCA analysis shows that reprogramming towards primed and naïve pluripotency clearly diverge at day 8 (FIG. 5d). For example, the naïve-state markers DPPA5 and KLF4 were robustly induced only under generation of naïve iPSCs. The primed-state markers DUSP6 and SPRY4 were initially induced under both conditions, but were maintained only in PRM, while the shared markers POU5F1 and PRDM14 followed similar kinetics under both conditions (FIG. 5d, 5e). Between day 5 and 8 cells undergo dramatic morphological changes indicative of a MET. A reduction in mesenchymal markers (FIG. 5f, in black) and gradual activation of epithelial markers (in grey) was revealed for both reprogramming protocols, indicating that MET is a common early molecular event. The here described reprograming method allows to study molecular changes and the distinct reprogramming trajectories leading to either primed or naïve pluripotency starting from human somatic cells.

Epigenetic and Metabolic Characterization of niPSCs

Naïve pluripotency is associated with a reduction in repressive epigenetic modification, such as trimethylation of lysine 9 on histone 3 (H3K9me3) or cytosine methylation (5mC). Both markers were found to be reduced by immunostaining in multiple niPSCs compared to primed PSCs (data not shown). Consistent with the reduction in 5mC, a decreased level of DNA methyltransferases DNMT3A and DNMT3B, a dramatic increase in the catalytically inactive DNMT3L, and increased expression of the 5mC oxidases TET1 and TET2 were observed (FIG. 6a).

To further study the impact of direct reprogramming to naïve pluripotency on DNA methylation, the pattern of genome methylation was determined by Reduced Representation Bisulfite Sequencing (RRBS) in 5 niPSC lines, 3 fibroblasts from which they were derived and an isogenic primed iPSC line. Reset H9 cells 21 were also included as a control. niPSCs had average global methylation levels (FIG. 6b) and distributions (FIG. 6c) comparable to those of human blastocysts and other naïve PSCs. Unsupervised clustering of the methylation pattern clearly separated naïve PSCs from somatic cells and primed iPSCs (FIG. 6d). Previous studies identified a set of promoters hypomethylated in human blastocysts compared to conventional primed hES cells and transiently hypomethylated during reprogramming in PRM (Smith, Z. D. et al. 2014 DNA methylation dynamics of the human preimplantation embryo. Nature 511, 611; Cacchiarelli, D. et al. 2015 Integrative Analyses of Human Reprogramming Reveal Dynamic Nature of Induced Pluripotency. Cell 162, 412-424). Interestingly, such loci were found highly methylated in somatic and primed pluripotent cells, and hypomethylated in niPSCs, in line with their naïve identity (FIG. 6e).

DNA methylation is crucial for genomic imprinting, the phenomenon causing expression of either the maternal or paternal copy of a gene. By analyzing 67 bona fide imprinted loci, 3 distinct behaviors were observed. A set of loci was devoid of DNA methylation in all samples (FIG. 6f, light grey). Such loci were defined “placental”, as they were found methylated in human oocytes and blastocysts, but not in somatic cells (blood). These data confirm that in somatic cells (fibroblasts) such loci are mostly hypomethylated and remained so also after reprogramming to either naïve or primed pluripotency. Conversely, DNA methylation at maternally imprinted loci was lost only in niPSCs and Reset H9 cells. Finally, a fraction (⅝) of paternally imprinted loci were surprisingly maintained in all niPSCs.

RNA-seq data were interrogated taking advantage of annotated Single Nucleotide Polymorphisms (SNPs) in mRNAs of interest. MEG3 showed monoallelic expression in somatic cells, and was biallelically expressed in all naive PSCs, in agreement with its reduced methylation (FIG. 6g). In sum, the here presented data indicate that niPSCs display a global DNA methylation profile consistent with naïve pluripotent cells and that the majority of imprinted loci are aberrantly hypomethylated in niPSCs, although a fraction of paternal loci are maintained.

Female naïve PSCs, as well as pluripotent cells from the human blastocysts, contain two active X chromosomes. Inactivation of the X chromosome is characterized by high levels of DNA methylation at CG-reach regions called CpG islands (CGIs). Levels of CGI methylation on X chromosome were measured in female niPSCs and a dramatic reduction was observed (FIG. 6h) relative to female fibroblasts, comparable to levels observed in male cells. Analysis of SNPs on Xlinked genes showed biallelic expression of several transcripts in female niPSCs (FIG. 6i).

Finally, high XIST expression was detected in female niPSCs, compared to male niPSCs and somatic cells (FIG. 6j), consistent with previous studies reporting expression of XIST from both active X chromosomes in female naïve pluripotent cells in vitro and in vivo. All these results indicate reactivation of both X chromosomes in female niPSCs.

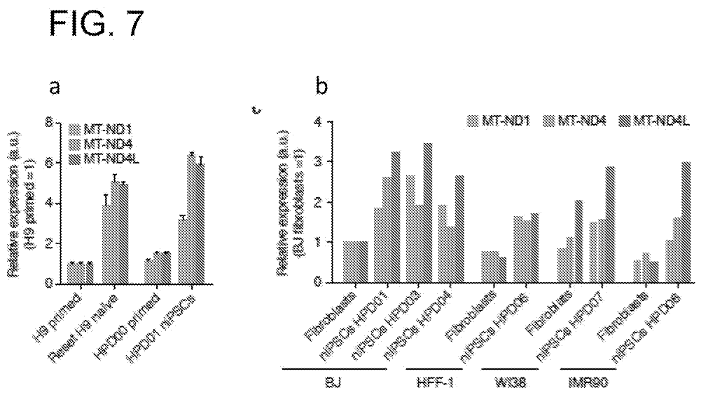

Mitochondrial activity has been reported to be higher in both murine and human naïve cells, compared to their primed counterparts. The mitochondrial membrane potential was measured by TMRM staining and it was barely detectable in primed cells (data not shown). Conversely, niPSCs displayed robust TMRM staining that co-localised with a mitochondrial dye (MitoTracker). Moreover, both niPSCs and Reset H9 cells 21 displayed a robust induction of mitochondrial transcripts, compared to primed PSCs (FIG. 7a) and to fibroblasts (FIG. 7b), as previously reported in murine PSCs.

niPSCs display epigenetic and metabolic features consistent with a naïve pluripotent state.

Characterization of the Differentiation Potential of niPSCs

From the literature, it is known that human naïve PSC lines are lineage biased and fail to differentiate towards mature cell types (Lee, J.-H. et al. 2017 Lineage-Specific Differentiation Is Influenced by State of Human Pluripotency. Cell Rep. 19, 20-35; Warrier, S. et al. 2017 Direct comparison of distinct naive pluripotent states in human embryonic stem cells. Nat. Commun. 8, 15055) thus limiting their use for disease modeling and developmental studies.

3 lines (HPD01, HPD03 and HPD04) generated from the same starting fibroblasts (BJ) were tested, including an isogenic primed iPSC line (HPD00) as a control.

First, 3 different monolayer differentiation protocols have been applied, and found that all 3 niPSC lines expressed markers of Mesoderm and Endoderm after 6 days of differentiation, while Ectoderm markers were robustly expressed around day 12. As expected primed iPSCs differentiated faster (data not shown).

Second, embryo body (EB) differentiation has been performed and expression of multiple markers of the three germ layers was detected at day 22 in all 3 niPSC lines (data not shown).

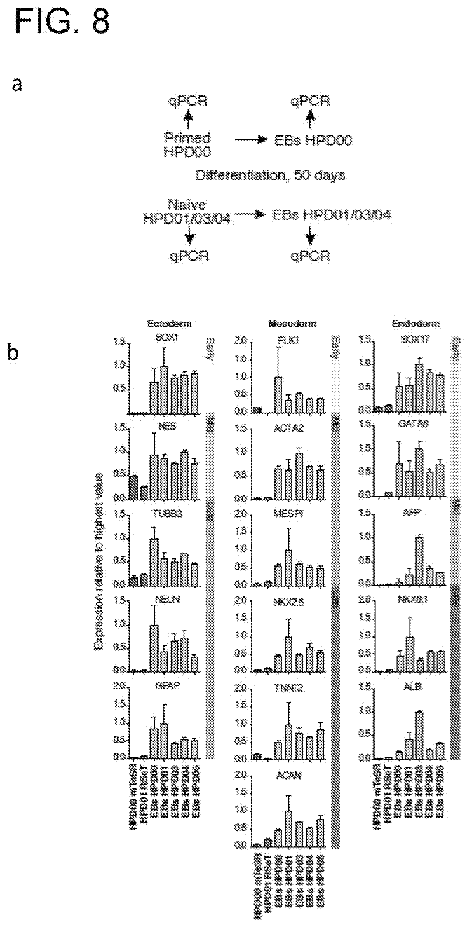

Third, the capacity to express markers of mature cell types after 50 days of EB differentiation was evaluated As shown in FIGS. 8a and 8b, all niPSCs differentiated as robustly as their isogenic primed counterpart, with expression of mature markers of the 3 germ layers. Fourth, the ability to form mature cell types, such as neurons and hepatocytes was tested. After 15 days of hepatic differentiation protocol, according to the protocols original described by Hay, D. C. et al. 2008 (Efficient differentiation of hepatocytes from human embryonic stem cells exhibiting markers recapitulating liver development in vivo. Stem Cells Dayt. Ohio 26, 894-902) and Hay, D. C. et al. 2008 (Highly efficient differentiation of hESCs to functional hepatic endoderm requires ActivinA and Wnt3a signaling. Proc. Natl. Acad. Sci. U.S.A 105, 12301-12306). Polygonal shaped hepatocyte-like cells expressing the mature markers CYP3A and HNF4A were observed, while the neuronal differentiation protocol according to Errichelli, L. et al. 2017 (FUS affects circular RNA expression in murine embryonic stem cell derived motor neurons. Nat. Commun. 8, 14741) successfully generated MAP2+, TUJ1+, NeuN+ cells.

Finally, all differentiation assays were successfully repeated with an independent niPSC line (HPD06) derived from HFF fibroblasts, further indicating robustness of the here described method.

In sum, these results demonstrate that niPSCs obtained according to the here described method are pluripotent, respond effectively to differentiation cues and are able to form mature cell types.

### The Confined Microfluidic Environment Promotes Establishment of Naïve Pluripotency

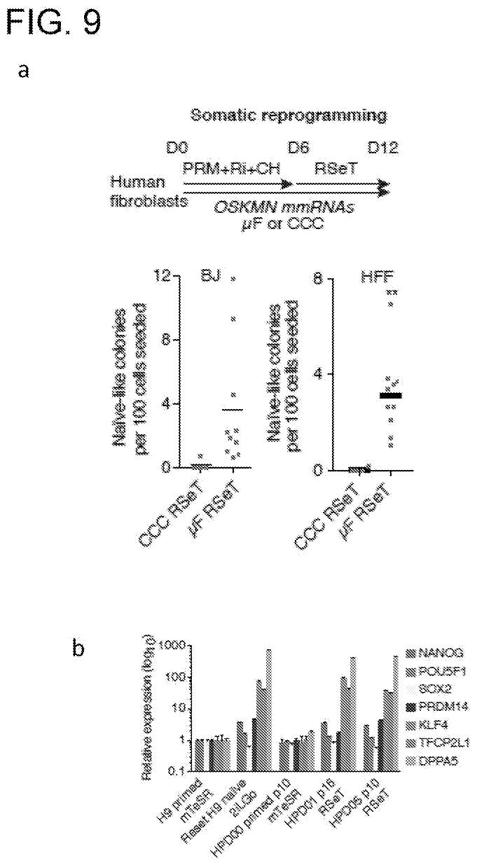

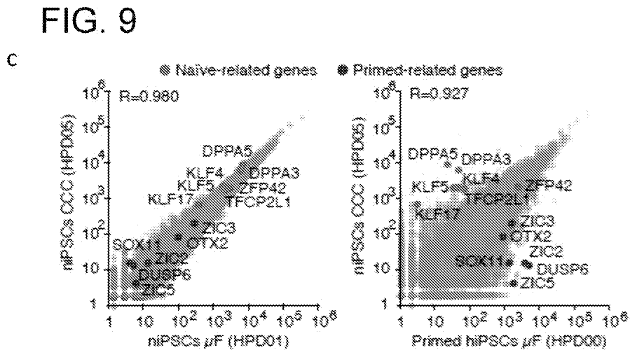

Side-by-side experiments comparing μF and CCC were performed. Generation of niPSCs in μF was robust (100% of channels contained colonies, with an efficiency of 3.7%+1.0 and 3.1%+0.4 from BJ and HFF-1, respectively, FIG. 9a). Conversely, in CCC only 25% of wells contained naïve-like colonies, with efficiency of 0.2% and 0.04%. Interestingly, a mixture of colonies displaying primed and naïve morphology emerged in CCC, a phenomenon never observed in μF. These results strongly indicate that the confined microenvironment promotes generation of naïve iPSCs over primed iPSCs. The mixture of primed- and naïve-like colonies generated in CCC was expanded and, by adding the FGF receptor inhibitor PD173074, primed iPS cells were eliminated. CCC-generated niPSCs (HPD05) were successfully expanded and their transcriptome was comparable to other naïve iPSCs and clearly distinct from primed PSCs (FIG. 9b, c).

To directly test if the confined microenvironment promotes reprogramming to the naïve state, microfluidics chips with channels of different heights were generated, inside which BJ fibroblasts were reprogrammed. At the height of 200 μm, used in all other experiments, colonies at the expected high efficiency (˜6 colonies per 100 cells seeded) have been obtained and all colonies expressed both KLF17 and POU5F1. Increasing the height caused a reduction in both the number of colonies, and in the percentage of double positive colonies. For instance, at 1000 μm height, only 44% of the colonies expressed both KLF17 and POU5F1, indicating the presence of either primed or partially reprogrammed colonies. Finally, decreasing the channel height to 100 μm caused a reduction in colony number, likely due to rapid exhaustion of the culture medium. Reprogramming to naïve pluripotency is therefore enhanced by an optimal spatial confinement.

Finally, over-expression of NANOG and KLF2 (NK2) was used to reset primed PSCs to the naïve state. DOX-inducible NK2 primed H9 cells were generated and exposed to 2iL+DOX, as previously reported. After 14 days, the cells obtained were stained for the naïve marker TFCP2L1. A 5-fold increase in the number of TFCP2L1 positive colonies in μF compared to CCC was observed. In contrast, under conditions promoting primed pluripotency (FGF/KSR) no TFCP2L1 positive colonies were observed even in μF. μF environment indeed promotes activation of the naïve pluripotency network, regardless of the starting cell type, but only in conditions supporting naïve pluripotency.

1. A method to derive naïve iPSCs (niPSCs) from mammalian somatic cells, said method comprising a transgene cocktail delivery to somatic cells in a microfluidic setting, and a first step of Mesenchymal to Epithelial Transition (MET) and a second step of colonies generation.

2. The method according to claim 1, said somatic cells being human somatic cells, selected from the group comprising fibroblast cells, epithelial cells, blood cells.

3. The method according to claim 1, wherein said cells are selected from the group comprising human foreskin fibroblasts HFF, lung fibroblasts WI-38 and IMR-90, tubular kidney cells, monocytes.

4. The method according to claim 1, said first step comprising incubating said somatic cells in Primed Reprogramming Medium (PRM) in the presence of a GSK inhibitor for 5 to 8 days.

5. The method according to claim 1, said second step comprising changing the medium into a medium for pluripotent cells, said second step being protracted for a period of between 4 and 10 days.

6. The method according to claim 1, wherein said transgene cocktail comprises nucleotidic acids sequences encoding for at least one of OCT4, SOX2, KLF4 and cMYC (OSKM) and NANOG, or a combination thereof.

7. The method according to claim 6, wherein said transgene cocktail comprises nucleotide acid sequences encoding for OSKM and NANOG.

8. The method according to claim 1, wherein every day during said first and said second step, cells are transfected with mmRNA encoding for OSKMN (OSKM+NANOG).

9. The method according to claim 1, wherein at day −1 said somatic cells are seeded, in DMEM+10% FBS, at 15-30 cells mm-2.

10. The method according to claim 8, wherein at day 0 the medium is changed to PRM medium, comprising E8, StemMACS ReproBREW XF (Miltenyi biotech) and cells are kept in the PRM medium from day 0 to day 5-8.

11. The method according to claim 1, wherein every day said PRM medium is changed and, about 12-17 h after said medium change, mmRNAs are added.

12. The method according to claim 1, wherein the transfection is protracted for about 4 to 12 h.

13. The method according to claim 1, said GSK inhibitor is selected from the group comprising: CHIR99021 (Stemcell technologies Inc.), SB-216763 (BIOMOL International), IM-12 (Enzo).

14. The method according to claim 13, said GSK inhibitor is CHIR99021 and CHIR99021 1 μM is added.

15. The method according to claim 1, further comprising, during said first step, the addition of a ROCK inhibitor, said ROCK inhibitor being Y-27632 (Sigma Aldrich) and being added at 5 μM of said inhibitor.

16. The method according to claim 1, said medium for pluripotent cells being selected from the group comprising N2B27, RSeT (Stemcell technologies Inc), 4iLA, 2iLGo.

17. The method according to claim 1, wherein every day in said second step said medium for pluripotent cells is changed and, about 12-17 h after said medium change, mmRNAs are added.

18. The method according to claim 1, wherein cells are maintained, at least for a fraction of the time of said first and said second step, in a 5% CO₂, 5% O₂ incubator.

19. The method according to claim 1, wherein said microfluidic platform channels are about 45 μm square in section and culture channel molds are about 220 μm square in section.

20. The method according to claim 1, wherein said cells are maintained from day 0 to day 6 in PRM medium and at day six said medium is changed into a medium for pluripotent cells, wherein the cells are kept from day 6 to day 12.

21. Naïve iPSCc (niPSCS) obtained according to the method of claim 1 which are highly clonogenic and have a high chromosomal stability.

22. Naïve iPSCc (niPSCS) obtained according to the method of claim 1 expressing naïve markers KLF17, TFCP2L1, DPPA and DNMT31L.

23. Naïve iPSCc (niPSCS) obtained according to the method of claim 1, which do not express SSEA4.
